(19) United States
(12) Patent Application Publication
(10) Pub. No.: US 2026/0035698 A1
(43) Pub. Date: Feb. 5, 2026

(54) COMPRISING EXOSOMAL MIR-664A-5P AS AN ACTIVE INGREDIENT, A DIAGNOSTIC BIOMARKER COMPOSITION FOR PARP INHIBITOR RESPONSE AND A PHARMACEUTICAL COMPOSITION FOR CANCER TREATMENT

(71) Applicant: THE CATHOLIC UNIVERSITY OF KOREA INDUSTRY-ACADEMIC COOPERATION FOUNDATION, Seoul (KR)

(72) Inventors: Ji Youl LEE, Seoul (KR); Mee Young KIM, Seoul (KR)

(21) Appl. No.: 19/283,918

(22) Filed: Jul. 29, 2025

(30) Foreign Application Priority Data
Jul. 30, 2024 (KR) 10-2024-0100631
Nov. 6, 2024 (KR) 10-2024-0156591

(51) Int. Cl. C12N 15/113; A61K 31/454; A61K 31/502; A61K 31/5025; A61K 31/55; A61K 31/7105; A61P 35/00; C12Q 1/6886

(52) U.S. Cl. CPC C12N 15/113; A61K 31/454; A61K 31/502; A61K 31/5025; A61K 31/55; A61K 31/7105; A61P 35/00; C12N 15/1137; C12Q 1/6886; C12N 2310/141; C12Q 2600/106; C12Q 2600/158; C12Q 2600/178

(57) ABSTRACT

The present invention relates to a diagnostic biomarker composition for PARP inhibitor response and a pharmaceutical composition for cancer treatment, including at least one miRNA selected from the group consisting of exosomal miR-664a-5p, miR-98-5p, and miR-95-3p as an active ingredient. The biomarker of the present invention can predict the therapeutic responsiveness and prognosis of cancer patients to PARP inhibitors, enabling the early identification of patients in whom the efficacy of PARP inhibitors is limited due to the development of resistance. Additionally, it has been confirmed that the biomarker of the present invention directly targets FOXM1, a transcription factor involved in chemotherapy resistance, to enhance sensitivity to PARP inhibitors. This finding demonstrates that the differential expression of the biomarker of the present invention plays a crucial role in regulating the efficacy of PARP inhibitors. Furthermore, the biomarker of the present invention can be identified as a potential therapeutic target for PARP inhibitor response in cancer patients.

Accordingly, the biomarker of the present invention, either alone or in combination with a PARP inhibitor, can be effectively utilized for the treatment, prevention, and improvement of cancer.

### CROSS REFERENCE TO RELATED APPLICATIONS

This application claims priority to Korean Patent Application No. 10-2024-0100631, filed on Jul. 30, 2024, and Korean Patent Application No. 10-2024-0156591, filed on Nov. 6, 2024, the entire contents of each of which are incorporated herein by reference.

### STATEMENT REGARDING FEDERALLY SPONSORED RESEARCH OR DEVELOPMENT

The present invention was made with the Individual Basic Research Program #2020R1A2C1102461 of the Ministry of Science and ICT and the Korea Medical Device Development Program #2020R1A2C1102461 of the Multi-Ministerial.

### REFERENCE TO A SEQUENCE LISTING SUBMITTED AS AN XML FILE

The Sequence Listing written in the XML file titled “206132-0194-00US_SEQUENCE LISTING.xml” in XML format, with a creation date of Jul. 17, 2025, and 14,877 bytes in size, is hereby incorporated by reference in its entirety.

### BACKGROUND

### 1. Field of the Invention

The present invention relates to a diagnostic biomarker composition for PARP inhibitor response and a pharmaceutical composition for cancer treatment, comprising exosomal miR-664a-5p as an active ingredient.

### 2. Background Art

Globally, the number of cancer patients has been increasing each year, and in South Korea, the number reached 100,000 in the year 2002. Among them, breast cancer, gastric cancer, colorectal cancer, ovarian cancer, liver cancer, prostate cancer, pancreatic cancer, and lung cancer exhibit the highest incidence rates worldwide. The treatment efficacy of these cancers varies depending on the early prediction of treatment responsiveness before administering anticancer therapy. Additionally, anticancer treatment, which can cost from several million to tens of millions of Korean won, may, in exceptional cases, cause adverse effects, and its therapeutic efficacy is not necessarily guaranteed for all cancer patients' tumors. Treatment with chemotherapeutic agents or targeted anticancer drugs can physically and/or mentally burden cancer patients. However, the therapeutic effects may not appear in all cancer patients, and resistance may also occur. Therefore, before administering immunotherapeutic agents or targeted anticancer drugs to cancer patients, it is crucial to determine whether these therapies will appropriately respond in the patient.

Meanwhile, microRNA (miRNA) is a small RNA molecule that primarily functions in regulating gene expression. Generally, miRNAs consist of approximately 21-23 nucleotide sequences and regulate protein expression within cells by degrading mRNA or inhibiting its translation. Due to the action of these miRNAs, cells can finely regulate gene expression in various physiological processes. Since miRNAs can regulate the expression of specific target genes, they have recently been actively studied as novel therapeutic candidates for various intractable diseases. Additionally, the relatively small size of miRNAs makes them easily adaptable to various delivery vehicles, allowing them to overcome the inherent vulnerability of RNA to degradation, making them a promising bioactive substance. The present inventors have completed this invention by confirming that miRNAs not only effectively inhibit cancer tumor growth and can be used as a therapeutic agent but also can be utilized in combination with other anticancer therapies as a treatment.

### PRIOR ART DOCUMENTS

### Patent Documents

(Patent Document 1) (Korean Published Patent) No. 10-2024-0040657 (Mar. 28, 2024)

### DISCLOSURE

### Technical Problem

The objective of the present invention is to provide a method for providing information for predicting therapeutic responsiveness or prognosis for a PARP (Poly (ADP-ribose) polymerase) inhibitor, including:
(S1) measuring the expression level of at least one miRNA gene selected from the group consisting of miR-664a-5p, miR-98-5p, and miR-95-3p from a biological sample isolated from a cancer patient;
(S2) comparing the expression level of the miRNA gene measured in step (S1) with the expression level of at least one miRNA gene selected from the group consisting of miR-664a-5p, miR-98-5p, and miR-95-3p in a biological sample isolated from a control group; and
(S3) predicting that the therapeutic responsiveness to a PARP inhibitor is high, or the prognosis is favorable, when the expression level of the miRNA gene measured in step (S1) is higher than the expression level of the miR-664a-5p gene in the biological sample isolated from the control group, or predicting that the therapeutic responsiveness to a PARP inhibitor is low, or the prognosis is poor, when the expression level of the miRNA gene measured in step (S1) is higher than the expression level of at least one miRNA gene selected from the group consisting of miR-98-5p and miR-95-3p in the biological sample isolated from the control group.

Another objective of the present invention is to provide a composition for predicting therapeutic responsiveness or prognosis for a PARP inhibitor, including an agent for measuring the expression level of at least one miRNA gene selected from the group consisting of miR-664a-5p, miR-98-5p, and miR-95-3p as an active ingredient.

Another objective of the present invention is to provide a kit for predicting therapeutic responsiveness or prognosis for a PARP inhibitor, including the composition of the present invention and an instruction manual.

Another objective of the present invention is to provide a pharmaceutical composition for the prevention or treatment of cancer, including at least one miRNA gene selected from the group consisting of miR-664a-5p, miR-98-5p, and miR-95-3p as an active ingredient.

Another objective of the present invention is to provide a kit for the prevention or treatment of cancer, including the composition of the present invention and an instruction manual.

Another objective of the present invention is to provide a method for screening an anticancer agent, including:
(S1) Treating a biological sample isolated from a cancer patient with a test substance;
(S2) Measuring the expression level of at least one miRNA gene selected from the group consisting of miR-664a-5p, miR-98-5p, and miR-95-3p in the biological sample treated with the test substance; and
(S3) Screening a test substance that increases the expression level of miR-664a-5p compared to a control sample, or screening a test substance that decreases the expression level of at least one miRNA gene selected from the group consisting of miR-98-5p and miR-95-3p compared to a control sample.

Another objective of the present invention is to provide a composition for screening anticancer agents, including an agent for measuring the expression level of at least one miRNA gene selected from the group consisting of miR-664a-5p, miR-98-5p, and miR-95-3p as an active ingredient.

However, the technical problems to be solved by the present invention are not limited to those mentioned above, and other problems not explicitly stated will be clearly understood by those of ordinary skill in the technical field to which the present invention pertains from the following description.

### Technical Solution

To achieve the above objectives, the present invention provides a method for providing information for predicting therapeutic responsiveness or prognosis for a PARP (Poly (ADP-ribose) polymerase) inhibitor, including:
(S1) Measuring the expression level of at least one miRNA gene selected from the group consisting of miR-664a-5p, miR-98-5p, and miR-95-3p from a biological sample isolated from a cancer patient;
(S2) Comparing the expression level of the miRNA gene measured in step (S1) with the expression level of at least one miRNA gene selected from the group consisting of miR-664a-5p, miR-98-5p, and miR-95-3p in a biological sample isolated from a control group; and
(S3) Predicting that the therapeutic responsiveness to a PARP inhibitor is high, or the prognosis is favorable, when the expression level of the miRNA gene measured in step (S1) is higher than the expression level of miR-664a-5p in the biological sample isolated from the control group, or
predicting that the therapeutic responsiveness to a PARP inhibitor is low, or the prognosis is poor, when the expression level of the miRNA gene measured in step (S1) is higher than the expression level of at least one miRNA gene selected from the group consisting of miR-98-5p and miR-95-3p in the biological sample isolated from the control group.

In one embodiment of the present invention, the method further comprises, after step (S3), administering to the cancer patient of step (S1) a PARP inhibitor, and/or at least one miRNA selected from the group consisting of miR-664a-5p, miR-98-5p, and miR-95-3p or an overexpression agent thereof, wherein the overexpression agent thereof refer to increasing the expression level of at least one miRNA gene selected from the group consisting of miR-664a-5p, miR-98-5p, and miR-95-3p, and may be a mimic, an expression construct, or an expression vector of the gene, but is not limited thereto.

In another embodiment of the present invention, the cancer in step (S1) may be prostate cancer, but is not limited thereto.

In another embodiment of the present invention, the biological sample in step (S1) may be at least one selected from the group consisting of urine, urine-derived exosomes, feces, feces-derived exosomes, saliva, and saliva-derived exosomes, but is not limited thereto.

In another embodiment of the present invention, the PARP inhibitor may be at least one selected from the group consisting of olaparib, talazoparib, niraparib, and rucaparib, but is not limited thereto.

The present invention provides a composition for predicting therapeutic responsiveness or prognosis for a PARP inhibitor, including an agent for measuring the expression level of at least one miRNA gene selected from the group consisting of miR-664a-5p, miR-98-5p, and miR-95-3p as an active ingredient.

In one embodiment of the present invention, the PARP inhibitor may be at least one selected from the group consisting of olaparib, talazoparib, niraparib, and rucaparib, but is not limited thereto.

In another embodiment of the present invention, the agent for measuring the expression level of the miRNA gene may be at least one agent selected from the group consisting of a primer and a probe that specifically bind to the gene, but is not limited thereto.

The present invention provides a kit for predicting therapeutic responsiveness or prognosis for a PARP inhibitor, including the composition of the present invention and an instruction manual.

In one embodiment of the present invention, the instruction manual may state that a cancer patient whose expression level of the miR-664a-5p gene is higher than the expression level of the miR-664a-5p gene in a biological sample isolated from a control group is predicted to have high therapeutic responsiveness to a PARP (Poly (ADP-ribose) polymerase) inhibitor or a favorable prognosis, or that a cancer patient whose expression level of at least one miRNA gene selected from the group consisting of miR-98-5p and miR-95-3p is higher than the expression level of at least one miRNA gene selected from the group consisting of miR-98-5p and miR-95-3p in a biological sample isolated from a control group is predicted to have low therapeutic responsiveness to a PARP inhibitor or a poor prognosis, but is not limited thereto.

In another embodiment of the present invention, the cancer may be prostate cancer, but is not limited thereto.

In another embodiment of the present invention, the kit may be at least one selected from the group consisting of a microarray, an aptamer chip kit, an ELISA (Enzyme-Linked Immunosorbent Assay) kit, a blotting kit, an immunoprecipitation kit, an immunofluorescence assay kit, a protein chip kit, a reverse transcription polymerase chain reaction (RT-PCR) kit, and a real-time polymerase chain reaction (qRT-PCR) kit, but is not limited thereto.

The present invention provides a pharmaceutical composition for the prevention or treatment of cancer, including at least one miRNA gene selected from the group consisting of miR-664a-5p, miR-98-5p, and miR-95-3p as an active ingredient.

In one embodiment of the present invention, the cancer may be prostate cancer, but is not limited thereto.

In another embodiment of the present invention, the composition may further include a PARP (Poly (ADP-ribose) polymerase) inhibitor or a pharmaceutically acceptable salt thereof as an active ingredient, but is not limited thereto.

In another embodiment of the present invention, PARP inhibitor may be at least one selected from the group consisting of olaparib, talazoparib, niraparib, and rucaparib, but is not limited thereto.

In another embodiment of the present invention, miR-664a-5p may be at least one selected from the group consisting of, but is not limited thereto:
(a) directly targeting FOXM1 (forkhead box M1);
(b) binding to the 3′UTR (3′ untranslated region) of FOXM1 mRNA;
(c) inhibiting the expression of the FOXM1 gene or the activity of the FOXM1 protein; and
(d) inhibiting the expression of at least one gene selected from the group consisting of BRCA2 (Breast Cancer Susceptibility Gene 2), BRIP1 (BRCA1-Interacting Protein 1), EXO1 (Exonuclease 1), and RAD51 (RAD51 Recombinase).

In another embodiment of the present invention, miRNA gene may be provided in a form loaded onto a carrier, but is not limited thereto.

The present invention provides a kit for the prevention or treatment of cancer, including the composition of the present invention and an instruction manual.

The present invention provides a method for screening an anticancer agent, including:
(S1) Treating a biological sample isolated from a cancer patient with a test substance;
(S2) Measuring the expression level of at least one miRNA gene selected from the group consisting of miR-664a-5p, miR-98-5p, and miR-95-3p in the biological sample treated with the test substance; and
(S3) Screening a test substance that increases the expression level of miR-664a-5p compared to a control sample, or screening a test substance that decreases the expression level of at least one miRNA gene selected from the group consisting of miR-98-5p and miR-95-3p compared to a control sample.

In one embodiment of the present invention, the cancer in step (S1) may be prostate cancer, but is not limited thereto.

In another embodiment of the present invention, the biological sample in step (S1) may be at least one selected from the group consisting of urine, urine-derived exosomes, feces, feces-derived exosomes, saliva, and saliva-derived exosomes, but is not limited thereto.

The present invention provides a composition for screening anticancer agents, including an agent for measuring the expression level of at least one miRNA gene selected from the group consisting of miR-664a-5p, miR-98-5p, and miR-95-3p as an active ingredient.

In one embodiment of the present invention, the agent for measuring the expression level of the miRNA gene may be at least one agent selected from the group consisting of primer and probe that specifically bind to the gene, but is not limited thereto.

The present invention provides a kit for screening anticancer agents, including the composition of the present invention and an instruction manual.

Furthermore, the present invention provides a method for preventing, ameliorating, alleviating, or treating cancer, including administering a pharmaceutically effective amount of a composition including at least one miRNA gene selected from the group consisting of miR-664a-5p, miR-98-5p, and miR-95-3p as an active ingredient to a subject in need thereof.

Furthermore, the present invention provides the use of a composition including at least one miRNA gene selected from the group consisting of miR-664a-5p, miR-98-5p, and miR-95-3p as an active ingredient for the prevention, amelioration, alleviation, or treatment of cancer.

Furthermore, the present invention provides the use of a composition including at least one miRNA gene selected from the group consisting of miR-664a-5p, miR-98-5p, and miR-95-3p as an active ingredient for the manufacture of a medicament for the prevention, amelioration, alleviation, or treatment of cancer.

### Advantageous Effects

The biomarker of the present invention can predict the therapeutic responsiveness and prognosis of cancer patients to PARP inhibitors, enabling the early identification of patients in whom the efficacy of PARP inhibitors is limited due to the development of resistance. In addition, it was confirmed that the biomarker of the present invention directly targets FOXM1, a transcription factor involved in chemotherapy resistance, thereby enhancing sensitivity to PARP inhibitors. This finding demonstrates that the differential expression of the biomarker of the present invention plays a crucial role in modulating the efficacy of PARP inhibitors. Furthermore, the biomarker of the present invention can be identified as a potential therapeutic target for PARP inhibitor response in cancer patients. Accordingly, the biomarker of the present invention, either alone or in combination with a PARP inhibitor, can be effectively utilized for the treatment, prevention, and improvement of cancer.

### BRIEF DESCRIPTION OF THE DRAWINGS

FIG. 1A shows the qRT-PCR primer sequences (SEQ ID NOs: 1 to 12), the FOXM1 mRNA 3′UTR, partial sequences of miR-664a-5p, and the sequences of miR-664a-5p mimic, miR-98-5p mimic, and miR-95-3p mimic (SEQ ID NOs: 13 to 17).

FIGS. 1B to 1D show Small RNA sequencing for the analysis of urinary exosomal miRNAs. FIG. 1B shows hierarchical clustering illustrating differentially expressed miRNAs in response to olaparib (fold-change>2, normalized read counts>6, p-value<0.05). FIG. 1C shows hierarchical clustering illustrating differentially expressed miRNAs in response to talazoparib (fold-change>2, normalized read counts>8). FIG. 1D shows a Venn diagram representing the intersections of the four miRNA expression profiles. Specifically, the red circle represents miRNAs that were found to be significantly upregulated in the response groups for both olaparib and talazoparib, while the blue circle represents miRNAs that were found to be significantly upregulated in the non-response groups for both olaparib and talazoparib.

FIGS. 2A to 2C show miR-664a-5p, miR-98-5p, and miR-95-3p, which increase sensitivity to PARP inhibitors in prostate cancer cells. Specifically, three prostate cancer cell lines (C4-2B, PC-3, and 22Rv1) were transfected with miR-664a-5p mimic (FIG. 2A), miR-98-5p mimic (FIG. 2B), miR-95-3p mimic (FIG. 2C), or negative control miRNA mimic (miR-NC), and then the cells were incubated with olaparib or talazoparib for 5 days. And cell viability was measured using the cell counting kit-8 assay. *P<0.05, **P<0.01, ***P<0.001. All data are presented as the mean±standard deviation (SD) of results obtained from two independent experiments performed in triplicate.

FIG. 2D shows miR-664a-5p, which enhances sensitivity to various PARP inhibitors. Specifically, C4-2B cells were transfected with miR-664a-5p mimic or negative control miRNA mimic (miR-NC) and then exposed to four different PARP inhibitors. After 5 days, cell viability was measured using the cell counting kit-8 assay. **P<0.01, ***P<0.001. The data are presented as the mean±standard deviation (SD) of results obtained from two independent experiments performed in triplicate.

FIGS. 3A to 3D show miR-664a-5p, which directly targets FOXM1. Specifically, FIG. 3A shows the schematic structure of the dual-luciferase reporter vector and the binding site between miR-664a-5p and FOXM1 predicted through miRWalk. FIG. 3B shows that PC-3 cells were co-transfected with the FOXM1 3′UTR-luciferase reporter and, either miR-664a-5p mimic or negative control miRNA mimic (miR-NC). Additionally, after 48 hours, the relative luciferase activity of the reporter was evaluated using a dual-luciferase reporter assay (3′UTR: 3′ untranslated region). FIGS. 3C and 3D show the results of transiently transfecting three prostate cancer cell lines (C4-2B, PC-3, and 22Rv1) with miR-664a-5p mimic or miR-NC mimic, followed by analysis of FOXM1 mRNA expression (FIG. 3C) and protein expression (FIG. 3D) after 3 days of transfection using qRT-PCR and western blot, respectively. β-actin was used as a loading control. **P<0.01, ***P<0.001. The data in FIGS. 3B and 3C are presented as the mean±standard deviation (SD) of results obtained from three independent experiments performed in triplicate.

FIGS. 4A to 4C show the combination of miR-664a-5p and olaparib, which reduces the expression of DNA repair genes. Specifically, C4-2B (FIG. 4A), PC-3 (FIG. 4B), and 22Rv1 (FIG. 4C) cells were transfected with either miR-664a-5p mimic or negative control miRNA mimic (miR-NC). The cells were then incubated with olaparib for 3 days, and mRNA expression was quantified using qRT-PCR. B2M mRNA was used as an internal control to normalize the data. *P<0.05, **P<0.01 vs. the olaparib group; #P<0.05, ##P<0.01, ###P<0.001 vs. the miR-664a-5p group. All data are presented as the mean±standard deviation (SD) of results obtained from two independent experiments performed in triplicate.

FIGS. 5A to 5C show the combination of miR-664a-5p and olaparib, which inhibits tumor growth in vivo. Specifically, FIG. 5A shows the timeline for the establishment of the PC-3 xenograft mouse model and the combination treatment. FIG. 5B shows the mean tumor volume of the four treatment groups (control, olaparib, miR-664a-5p, and combination) in the PC-3 xenograft model. FIG. 5C shows representative immunohistochemistry results for Ki-67 in the four treatment groups. Tumor tissues were stained using Ki-67 (brown), and nuclei were counterstained with hematoxylin (blue). The bar graph represents the quantitative assessment of Ki-67 expression. The scale bar represents 100 μm. **P<0.01, ***P<0.001. The data are presented as the mean±SE (n=6).

### MODES OF INVENTION

The inventors aimed to identify urinary exosomal miRNAs that are differentially expressed in response to PARP inhibitors in metastatic castration-resistant prostate cancer (mCRPC) patients and to investigate their potential as therapeutic targets. It was hypothesized that specific miRNAs could influence the sensitivity of prostate cancer cells to PARP inhibitors by regulating the expression of genes involved in the DNA repair pathway. Furthermore, the molecular mechanisms underlying the interaction between the identified miRNAs and PARP inhibitor response were explored, and the potential role of miRNAs in regulating the expression of DDR (DNA damage response) genes and forkhead box M1 (FOXM1) was identified. The present invention provides a method for providing information for predicting therapeutic responsiveness or prognosis for a PARP inhibitor, including:
(S1) Measuring the expression level of at least one miRNA gene selected from the group consisting of miR-664a-5p, miR-98-5p, and miR-95-3p from a biological sample isolated from a cancer patient;
(S2) Comparing the expression level of the miRNA gene measured in step (S1) with the expression level of at least one miRNA gene selected from the group consisting of miR-664a-5p, miR-98-5p, and miR-95-3p in a biological sample isolated from a control group; and
(S3) Predicting that the therapeutic responsiveness to a PARP (Poly (ADP-ribose) polymerase) inhibitor is high, or the prognosis is favorable, when the expression level of the miRNA gene measured in step (S1) is higher than the expression level of miR-664a-5p in the biological sample isolated from the control group, or
predicting that the therapeutic responsiveness to a PARP inhibitor is low, or the prognosis is poor, when the expression level of the miRNA gene measured in step (S1) is higher than the expression level of at least one miRNA gene selected from the group consisting of miR-98-5p and miR-95-3p in the biological sample isolated from the control group.

In the present invention, the cancer may be prostate cancer, breast cancer, gastric cancer, colorectal cancer, ovarian cancer, liver cancer, prostate cancer, pancreatic cancer, or lung cancer, but is not limited thereto.

In one embodiment of the present invention, the cancer in step (S1) may be prostate cancer, but is not limited thereto. In the present invention, prostate cancer (PCa) is the most common cancer in men worldwide and the fifth leading cause of cancer-related mortality. In addition, despite advancements in prostate cancer treatment, patients with advanced or metastatic disease often develop resistance to conventional therapies, which may lead to a poor prognosis.

In the present invention, the term “biological sample” may be at least one selected from the group consisting of urine, urine-derived exosomes, feces, feces-derived exosomes, saliva, saliva-derived exosomes, blood, serum, whole blood, plasma, tissue, cells, cancer cells, prostate cancer cells, organs, bone marrow, fine-needle aspiration biopsy samples, core needle biopsy samples, and vacuum-assisted biopsy samples, but is not limited thereto.

Additionally, in the present invention, the term “biological sample” may be used interchangeably with sample, but is not limited thereto.

Additionally, in the present invention, the term “biological sample” may be a biological sample isolated from a cancer patient, but is not limited thereto. Additionally, in the present invention, the term “isolated biological sample” may be obtained before, after, or regardless of the course of PARP inhibitor treatment, but is not limited thereto.

In one embodiment of the present invention, the biological sample in step (S1) may be at least one selected from the group consisting of urine, urine-derived exosomes, feces, feces-derived exosomes, saliva, and saliva-derived exosomes, but is not limited thereto.

In the present invention, the urine sample may be separated by centrifugation at a speed of 1000 to 4000 rpm, 1000 to 3900 rpm, 1000 to 3800 rpm, 1000 to 3700 rpm, 1000 to 3800 rpm, 1000 to 3700 rpm, 1000 to 3600 rpm, 1000 to 3500 rpm, 1000 to 3400 rpm, 1000 to 3300 rpm, 1000 to 3200 rpm, 1000 to 3100 rpm, 1000 to 3000 rpm, 1000 to 2900 rpm, 1000 to 2800 rpm, 1000 to 2700 rpm, 1000 to 2600 rpm, 1000 to 2500 rpm, 1500 to 4000 rpm, 1500 to 3900 rpm, 1500 to 3800 rpm, 1500 to 3700 rpm, 1500 to 3600 rpm, 1500 to 3500 rpm, 1500 to 3400 rpm, 1500 to 3300 rpm, 1500 to 3200 rpm, 1500 to 3100 rpm, 1500 to 3000 rpm, 1500 to 2900 rpm, 1500 to 2800 rpm, 1500 to 2700 rpm, 1500 to 2600 rpm, 1500 to 2500 rpm, 2000 to 4000 rpm, 2000 to 3900 rpm, 2000 to 3800 rpm, 2000 to 3700 rpm, 2000 to 3600 rpm, 2000 to 3500 rpm, 2000 to 3400 rpm, 2000 to 3300 rpm, 2000 to 3200 rpm, 2000 to 3100 rpm, 2000 to 3000 rpm, 2000 to 2900 rpm, 2000 to 2800 rpm, 2000 to 2700 rpm, 2000 to 2600 rpm, or 2000 to 2500 rpm. Specifically, the urine sample of the present invention may be separated by centrifugation at a speed of 2,500 rpm, but is not limited thereto.

In the present invention, the urine sample may refer to the supernatant extracted after centrifugation, but is not limited thereto.

In the present invention, urinary exosomal miRNA may serve as a therapeutic target and predictive biomarker for PARP inhibitor response in prostate cancer, but is not limited thereto. In addition, in the present invention, the use of urinary exosomal miRNA as a biomarker may be preferred in the related technical field due to the non-invasive nature of urine sampling. Since it may provide an advantage in the relevant technical field over other tissue-based biomarkers that require more invasive collection methods, the non-invasiveness of the biomarker of the present invention is consistent with the principles of precision medicine and can reduce patient discomfort while providing personalized therapeutic strategies, but is not limited thereto.

In the present invention, microRNA (miRNA) may be a small non-coding RNA that regulates post-transcriptional gene expression by binding to the 3′ untranslated region (UTR) of target mRNA. In addition, in the present invention, miRNA may play a crucial role in prostate cancer progression, metastasis, and therapeutic resistance, and circulating miRNA in blood or urine may serve as a non-invasive biomarker for prostate cancer diagnosis and prognosis, but is not limited thereto.

In the present invention, “miRNA (microRNA)” refers to a single-stranded RNA molecule consisting of 21-25 nucleotides that binds to the 3′-UTR of messenger RNA (mRNA) to regulate gene expression in eukaryotic organisms (Bartel DP, et al., Cell, 23;116(2): 281-297 (2004)). The biogenesis of miRNA involves the processing of a stem-loop structured precursor miRNA (pre-miRNA) by Drosha (an RNase III-type enzyme), followed by its transportation to the cytoplasm, where it is cleaved by Dicer to generate a mature miRNA. In the present invention, the miRNA may specifically include a nucleotide sequence selected from the group consisting of Sequence Nos. 14 to 17, and preferably, may consist of at least one nucleotide sequence selected from the group consisting of Sequence Nos. 14 to 17.

In addition, miR-664a-5p, miR-98-5p, and miR-95-3p used in the present invention encompass functional equivalents of the nucleic acid molecules constituting them, such as variants in which a part of the nucleotide sequence of the miRNA nucleic acid molecule is deleted, substituted, or inserted, as long as they can exert a functionally equivalent action to the miRNA nucleic acid molecule. For example, the miR-664a-5p, miR-98-5p, and miR-95-3p of the present invention may exhibit at least 80% sequence homology with the nucleotide sequences of SEQ ID NOs: 15, 16, and 17, respectively. Specifically, they may exhibit at least 90% homology, and more specifically, at least 95% homology. Such homology can be readily determined by comparing the nucleotide sequence with the corresponding region of the target gene using computer algorithms widely known in the field, such as the Align or BLAST algorithms.

Additionally, the miR-664a-5p, miR-98-5p, or miR-95-3p of the present invention may exist in a single-stranded or double-stranded form. A mature miRNA molecule primarily exists in a single-stranded form; however, a precursor miRNA molecule may include a partially self-complementary structure (e.g., a stem-loop structure) capable of forming a double strand. In addition, the nucleic acid molecule of the present invention may be composed in the form of RNA or peptide nucleic acids (PNA).

Additionally, the miR-664a-5p, miR-98-5p, or miR-95-3p of the present invention may be isolated or synthesized using standard molecular biology techniques, such as chemical synthesis or recombinant methods, or may be commercially available.

The miR-664a-5p, miR-98-5p, or miR-95-3p of the present invention may be the miR-664a-5p, miR-98-5p, or miR-95-3p itself or may include functionally equivalent fragments thereof. The fragment of the miRNA may be a polynucleotide including the seed sequence of the miRNA. The seed sequence refers to a nucleotide sequence within a region of the miRNA that binds to a target with complete complementarity when recognizing a target, and it is an essential segment required for the miRNA to bind to its target.

Additionally, the miR-664a-5p, miR-98-5p, or miR-95-3p can be used in the form of various miRNA mimics that induce biologically equivalent efficacy. Modified miRNA sequences including the same seed region as the original miRNA sequence can be utilized. As an miRNA mimic for the miRNA, it may partially include a phosphorothiolate structure, in which the RNA phosphate backbone structure is substituted with other elements such as sulfur. It can also be used in a fully or partially substituted form with DNA or PNA (peptide nucleic acids) molecules instead of RNA. Additionally, it can be used in a form where the 2′-hydroxyl group of the RNA sugar is substituted with various functional structures, including but not limited to methylation, methoxylation, and fluorination.

Additionally, in the present invention, the miR-664a-5p, miR-98-5p, or miR-95-3p may be in a form loaded onto extracellular vesicles, included in a vector, or introduced into a cell.

The miRNA of the present invention may be included in a pharmaceutically acceptable salt form. In the present invention, the term “pharmaceutically acceptable salt” includes salts derived from pharmaceutically acceptable inorganic acids, organic acids, or bases.

In the present invention, miR-664a-5p may be utilized as a biomarker for cancer diagnosis, as plasma miR-664a-5p levels have been associated with lung cancer risk. Additionally, miR-664a-5p may be involved in the regulation of cellular processes such as neuronal differentiation, apoptosis, mitochondrial homeostasis in vascular smooth muscle cells, and osteogenic differentiation of human bone marrow-derived mesenchymal stem cells. Furthermore, it may exert a broader influence on cellular mechanisms related to cancer progression, including cell differentiation, apoptosis, and cellular homeostasis, but is not limited thereto.

In the present invention, the step of measuring the expression level of the miRNA gene of the invention may be performed using at least one selected from the group consisting of a microarray, an aptamer chip kit, an ELISA (Enzyme-Linked Immunosorbent Assay) kit, a blotting kit, an immunoprecipitation kit, an immunofluorescence assay kit, a protein chip kit, a reverse transcription polymerase chain reaction (RT-PCR) kit, and a real-time polymerase chain reaction (qRT-PCR) kit, but is not limited thereto. In addition, in one embodiment of the present invention, the step of measuring the expression level of the miRNA gene of the invention may be performed by measuring the expression level of the miRNA gene through small RNA sequencing and may be performed by hierarchical clustering analysis, but is not limited thereto.

In the present invention, the term “control group” may refer to a person with and/or without cancer or another organism, wherein the organism may be a mammal such as a non-human primate, a mouse, rat, dog, cat, horse, or cow.

In the present invention, the term “patient” may refer to a person suspected of having and/or diagnosed with cancer, or another organism, wherein the organism may be a mammal such as a non-human primate, mouse, rat, dog, cat, horse, or cow. In addition, the term “patient” may be used interchangeably with “subject” or “individual,” but is not limited thereto.

In the present invention, the term “patient” refers to a subject in need of diagnosis, therapeutic responsiveness assessment, or prognosis prediction for a disease, and more specifically, it refers to a human or a mammal such as a non-human primate, mouse, rat, dog, cat, horse, or cow. In addition, in the present invention, the term “patient” may refer to a patient with high or low therapeutic responsiveness to a PARP inhibitor or a responder or non-responder to a PARP inhibitor, but is not limited thereto.

In the present invention, the term “comparison” may refer to individually comparing each miRNA selected from the group consisting of miR-664a-5p, miR-98-5p, and miR-95-3p, but is not limited thereto. Additionally, the comparison is performed using any method commonly used in the art and is a broad concept that includes both quantitative and qualitative comparisons.

In this specification, “high expression level” can be interchangeably used with “increased expression level.” In this context, “high expression level” refers to either the expression of a previously unexpressed gene or a relative increase in expression levels compared to the normal expression level. For example, an “increase” in the expression level means that the expression level of the miRNA gene in a sample is at least 1%, 2%, 3%, 4%, 5%, 10%, or more, such as 5%, 10%, 20%, 30%, 40%, 50%, 60%, 70%, 80%, 90%, or more higher compared to that of a control group, and/or at least 0.5-fold, 1.1-fold, 1.2-fold, 1.4-fold, 1.6-fold, 1.8-fold, or more higher.

Specifically, it may refer to an increase of 1 to 1.5-fold, 1.5 to 2-fold, 2 to 2.5-fold, 2.5 to 3-fold, 3 to 3.5-fold, 3.5 to 4-fold, 4 to 4.5-fold, 4.5 to 5-fold, 5 to 5.5-fold, 5.5 to 6-fold, 6 to 6.5-fold, 6.5 to 7-fold, 7 to 7.5-fold, 7.5 to 8-fold, 8 to 8.5-fold, 8.5 to 9 -fold, 9 to 9.5-fold, 9.5 to 10-fold, or more than 10-fold compared to that of the control group, but is not limited thereto. Additionally, in the present invention, “high miRNA gene expression level” may have a broad meaning, including a statistically significant level, but is not limited thereto. The opposite term thereof can be understood by those skilled in the art as having the opposite meaning in accordance with the above definition.

In the present invention, the term “analysis” can be used interchangeably with “identification,” “measurement,” or “detection” (and vice versa), and may include quantifying the content of a detected or measured target. Since it also encompasses the qualitative meaning of determining the presence or absence of a specific substance, it includes both measuring and confirming the presence (expression) of a target substance, as well as measuring and confirming changes in the level of presence (expression level) of the target substance.

In the present invention, the differential expression of at least one miRNA gene selected from the group consisting of miR-664a-5p, miR-98-5p, and miR-95-3p in response and non-response groups to a PARP inhibitor may indicate that the miRNA of the present invention plays an important role in regulating the efficacy of the PARP inhibitor, but is not limited thereto. Specifically, in the present invention, the differential expression of the miR-664a-5p gene in response and non-response groups to a PARP inhibitor may indicate that the miR-664a-5p of the present invention plays a crucial role in regulating the efficacy of the PARP inhibitor, but is not limited thereto.

In the present invention, the PARP inhibitor of the present invention may be at least one selected from the group consisting of olaparib, talazoparib, niraparib, rucaparib, veliparib, and pamiparib, but is not limited thereto.

Specifically, in one embodiment of the present invention, the PARP inhibitor of the present invention may be at least one selected from the group consisting of olaparib, talazoparib, niraparib, and rucaparib, but is not limited thereto.

In the present invention, poly (ADP-ribose) polymerase (PARP) inhibitors have emerged as a promising therapeutic strategy for prostate cancer, particularly in patients with defects in DNA damage response (DDR) genes such as BRCA1/2.

However, the clinical efficacy of PARP inhibitors is limited due to the development of resistance, creating a technical challenge to identify therapeutic targets that can enhance the response to these agents and overcome these limitations. Therefore, identifying biomarkers that can predict the response to PARP inhibitors and serve as potential therapeutic targets is a highly significant technical challenge for improving treatment outcomes and developing personalized therapies for prostate cancer patients.

In the present invention, “predicting that the therapeutic responsiveness to a PARP inhibitor is low or the prognosis is poor” may refer to “determining that the therapeutic responsiveness to a PARP inhibitor is low or the risk of cancer progression is high,” “determining that the therapeutic responsiveness to a PARP inhibitor is low or the risk of cancer occurrence is high,” “determining that the therapeutic responsiveness to a PARP inhibitor is low or the risk of cancer deterioration is high,” or “determining that the therapeutic responsiveness to a PARP inhibitor is low or the risk of cancer metastasis is high,” but is not limited thereto. The opposite term of this expression can be understood by a person skilled in the art as having the opposite meaning in accordance with the above definition. In the present invention, the phrase “predicted to have low therapeutic responsiveness or poor prognosis” can be used interchangeably with “determined to be a treatment-resistant patient,” but is not limited thereto.

In the present invention, “prediction of therapeutic responsiveness or prognosis” can be used interchangeably with “determining whether there is sensitivity or resistance to treatment” and “method for providing information on diagnosing resistance,” but is not limited thereto.

In the present invention, the “method for providing information on predicting therapeutic responsiveness or prognosis for a PARP inhibitor” may be a method for providing information on predicting the therapeutic responsiveness or prognosis of a cancer patient to a PARP inhibitor, but is not limited thereto.

In one embodiment of the present invention, “predicting therapeutic responsiveness or prognosis” may refer to the correlation between a PARP inhibitor and survival outcomes, but is not limited thereto.

In the present invention, “prediction of therapeutic responsiveness” may include predicting whether the prognosis will be favorable or unfavorable upon treatment with a PARP inhibitor, but is not limited thereto.

In the present invention, “prognosis prediction” may refer to the prediction of a cancer patient's prognosis, but is not limited thereto.

In the present invention, “prediction” may be used interchangeably with “risk prediction” and “risk assessment,” but is not limited thereto.

The present invention provides a composition for predicting therapeutic responsiveness or prognosis for a PARP inhibitor, including an agent for measuring the expression level of at least one miRNA gene selected from the group consisting of miR-664a-5p, miR-98-5p, and miR-95-3p as an active ingredient.

In the present invention, the PARP inhibitor of the present invention may be at least one selected from the group consisting of olaparib, talazoparib, niraparib, rucaparib, veliparib, and pamiparib, but is not limited thereto.

Specifically, in one embodiment of the present invention, the PARP inhibitor may be at least one selected from the group consisting of olaparib, talazoparib, niraparib, and rucaparib, but is not limited thereto.

In one embodiment of the present invention, the agent for measuring the expression level of the miRNA gene may be at least one selected from the group consisting of primers and probes that specifically bind to the gene, but is not limited thereto.

The present invention provides a kit for predicting therapeutic responsiveness or prognosis for a PARP inhibitor, including the composition of the present invention and an instruction manual.

In one embodiment of the present invention, the instruction manual may state that a cancer patient whose expression level of the miR-664a-5p gene is higher than the expression level of miR-664a-5p in a biological sample isolated from a control group is predicted to have high therapeutic responsiveness to a PARP (Poly (ADP-ribose) polymerase) inhibitor or a favorable prognosis; or that a cancer patient whose expression level of at least one miRNA gene selected from the group consisting of miR-98-5p and miR-95-3p is higher than the expression level of at least one miRNA gene selected from the group consisting of miR-98-5p and miR-95-3p in a biological sample isolated from a control group is predicted to have low therapeutic responsiveness to a PARP inhibitor or a poor prognosis, but is not limited thereto.

In one embodiment of the present invention, the instruction manual of the present invention may describe that a cancer patient whose expression level of the miRNA gene is higher than the expression level of the miR-664a-5p gene in a biological sample isolated from a control group is predicted to have high therapeutic responsiveness to a PARP (Poly (ADP-ribose) polymerase) inhibitor or a favorable prognosis; or that a cancer patient whose expression level of the miRNA gene is higher than the expression level of at least one miRNA gene selected from the group consisting of miR-98-5p and miR-95-3p in a biological sample isolated from a control group is predicted to have low therapeutic responsiveness to a PARP inhibitor or a poor prognosis; but is not limited thereto.

In the present invention, the cancer of the present invention may be prostate cancer, breast cancer, gastric cancer, colorectal cancer, ovarian cancer, liver cancer, prostate cancer, pancreatic cancer, and lung cancer, but is not limited thereto.

In one embodiment of the present invention, the cancer of the present invention may be prostate cancer, but is not limited thereto.

In one embodiment of the present invention, the kit of the present invention may be at least one selected from the group consisting of a microarray, an aptamer chip kit, an ELISA (Enzyme-Linked Immunosorbent Assay) kit, a blotting kit, an immunoprecipitation kit, an immunofluorescence assay kit, a protein chip kit, a reverse transcription polymerase chain reaction (RT-PCR) kit, and a real-time polymerase chain reaction (qRT-PCR) kit, but is not limited thereto.

The present invention provides a method for screening an anticancer agent, including:
(S1) Treating a biological sample isolated from a cancer patient with a test substance;
(S2) measuring the expression level of at least one miRNA gene selected from the group consisting of miR-664a-5p, miR-98-5p, and miR-95-3p in the biological sample treated with the test substance; and
(S3) screening a test substance that increases the expression level of miR-664a-5p compared to a control sample, or screening a test substance that decreases the expression level of at least one miRNA gene selected from the group consisting of miR-98-5p and miR-95-3p compared to a control sample.

The present invention provides a method for screening an anticancer agent, including:
(S1) Treating a biological sample isolated from a cancer patient with a test substance;
(S2) measuring the expression level of at least one miRNA gene selected from the group consisting of miR-664a-5p, miR-98-5p, and miR-95-3p in the biological sample treated with the test substance; and
(S3) screening a test substance that increases the expression level of the miRNA gene compared to a control sample.

In one embodiment of the present invention, the cancer in step (S1) may be prostate cancer, but is not limited thereto.

In another embodiment of the present invention, the biological sample in step (S1) may be at least one selected from the group consisting of urine, urine-derived exosomes, feces, feces-derived exosomes, saliva, saliva-derived exosomes, tissue, cells, cancer cells, and prostate cancer cells, but is not limited thereto.

In the present invention, the “biological sample” may be at least one selected from the group consisting of urine, urine-derived exosomes, feces, feces-derived exosomes, saliva, saliva-derived exosomes, blood, serum, whole blood, plasma, tissue, cells, cancer cells, prostate cancer cells, organs, bone marrow, fine-needle aspiration specimens, core needle biopsy specimens, and vacuum-assisted biopsy specimens, but is not limited thereto.

The present invention provides a composition for screening anticancer agents, including an agent for measuring the expression level of at least one miRNA gene selected from the group consisting of miR-664a-5p, miR-98-5p, and miR-95-3p as an active ingredient.

In one embodiment of the present invention, the agent for measuring the expression level of the miRNA gene may be at least one agent selected from the group consisting of a primer and a probe that specifically bind to the gene, but is not limited thereto.

The present invention provides a kit for screening anticancer agents, including the composition of the present invention and an instruction manual.

In one embodiment of the present invention, the instruction manual may state that a test substance is screened by identifying an increase in the expression level of miR-664a-5p compared to a control sample, or by identifying a decrease in the expression level of at least one miRNA gene selected from the group consisting of miR-98-5p and miR-95-3p compared to a control sample, but is not limited thereto.

In another embodiment of the present invention, the screened test substance may be determined to be an anticancer agent, but is not limited thereto.

In the present invention, the term “test substance” refers to an unknown substance used in screening to examine whether it affects the expression level of at least one miRNA gene selected from the group consisting of miR-664a-5p, miR-98-5p, and miR-95-3p in a biological sample. The test substance includes small interfering RNA (siRNA), short hairpin RNA (shRNA), microRNA (miRNA), ribozyme, DNAzyme, peptide nucleic acids (PNA), antisense oligonucleotides, recombinant plasmids, nanoparticles, proteins, oligopeptides, antibodies, aptamers, natural extracts, or chemical compounds, but is not limited thereto. In the present invention, the “test substance” may be used interchangeably with a candidate substance for an anticancer agent, but is not limited thereto.

In the present invention, treating the test substance may mean adding the test substance to a cell or tissue culture medium and then incubating the biological sample for a certain period, or bringing it into contact with the biological sample, but is not limited thereto. When the biological sample is provided in the form of a laboratory animal, contact with the test substance may include, but is not limited to, parenteral or oral administration and stereotactic injection. A person skilled in the art would be able to select an appropriate method to test the test substance in the animal.

The term “control group,” as used in reference to the screening method, may refer to a sample that has not been treated with the test substance, but is not limited thereto.

The present invention provides a method for screening an anticancer agent, including:
(S1) Treating a test substance in cancer cells;
(S2) Measuring the expression level of at least one miRNA gene selected from the group consisting of miR-664a-5p, miR-98-5p, and miR-95-3p in the biological sample treated with the test substance; and
(S3) Screening a test substance that increases the expression level of miR-664a-5p compared to a control sample, or screening a test substance that decreases the expression level of at least one miRNA gene selected from the group consisting of miR-98-5p and miR-95-3p compared to a control sample.

The present invention provides an in vitro method for screening anticancer agents, including:

(S1) Treating a test substance in cancer cells;

(S2) Measuring the expression level of at least one miRNA gene selected from the group consisting of miR-664a-5p, miR-98-5p, and miR-95-3p in the biological sample treated with the test substance; and

(S3) Screening a test substance that increases the expression level of miR-664a-5p compared to a control sample, or
screening a test substance that decreases the expression level of at least one miRNA gene selected from the group consisting of miR-98-5p and miR-95-3p compared to a control sample.

The present invention provides a method for screening an anticancer agent, including the steps of treating an isolated cancer cell with a candidate of anticancer agent and comparing the expression level of at least one miRNA gene selected from the group consisting of miR-664a-5p, miR-98-5p, and miR-95-3p.

The present invention provides a method for screening an anticancer agent, including the steps of treating a cancer cell with a candidate of anticancer agent and comparing the expression level of at least one miRNA gene selected from the group consisting of miR-664a-5p, miR-98-5p, and miR-95-3p.

The present invention provides a method for treating cancer, including the following steps:
(S1) Treating a biological sample isolated from a cancer patient with a test substance;
(S2) Measuring the expression level of at least one miRNA gene selected from the group consisting of miR-664a-5p, miR-98-5p, and miR-95-3p in the biological sample treated with the test substance;
(S3) Selecting a test substance that increases the expression level of miR-664a-5p compared to a control sample, or selecting a test substance that decreases the expression level of at least one miRNA gene selected from the group consisting of miR-98-5p and miR-95-3p compared to a control sample; and
(S4) Administering the selected test substance to the cancer patient of step (S1) for treatment.

The present invention provides a method for treating cancer, including the following steps:
(S1) Treating a biological sample isolated from a cancer patient with a test substance;
(S2) Measuring the expression level of at least one miRNA gene selected from the group consisting of miR-664a-5p, miR-98-5p, and miR-95-3p in the biological sample treated with the test substance;
(S3) Selecting a test substance that increases the expression level of miR-664a-5p compared to a control sample, or selecting a test substance that decreases the expression level of at least one miRNA gene selected from the group consisting of miR-98-5p and miR-95-3p compared to a control sample; and
(S4) Administering the selected test substance and a PARP (Poly (ADP-ribose) polymerase) inhibitor to the cancer patient of step (S1) for treatment.

In the present invention, the PARP (Poly (ADP-ribose) polymerase) inhibitor of the invention may be at least one selected from the group consisting of olaparib, talazoparib, niraparib, rucaparib, veliparib, and pamiparib, but is not limited thereto. Specifically, in one embodiment of the present invention, the PARP=inhibitor of the invention may be at least one selected from the group consisting of olaparib, talazoparib, niraparib, and rucaparib, but is not limited thereto.

The present invention provides a pharmaceutical composition for the prevention or treatment of cancer, including at least one miRNA gene selected from the group consisting of miR-664a-5p, miR-98-5p, and miR-95-3p as an active ingredient.

In the present invention, at least one miRNA gene selected from the group consisting of miR-664a-5p, miR-98-5p, and miR-95-3p may be derived from at least one selected from the group consisting of urine, urine-derived exosomes, feces, feces-derived exosomes, saliva, saliva-derived exosomes, tissues, cells, cancer cells, and prostate cancer cells, but is not limited thereto.

In one embodiment of the present invention, urinary exosomal miR-664a-5p was identified as a potential therapeutic target for the PARP inhibitor response in prostate cancer patients. The synergistic effect of miR-664a-5p and olaparib in suppressing prostate cancer tumor growth provided a basis for developing miRNA-based therapies in combination with PARP inhibitors for prostate cancer treatment.

In the present invention, the cancer of the present invention may be prostate cancer, breast cancer, gastric cancer, colorectal cancer, ovarian cancer, liver cancer, prostate cancer, pancreatic cancer, or lung cancer, but is not limited thereto.

In one embodiment of the present invention, the cancer of the present invention may be prostate cancer, but is not limited thereto.

In one embodiment of the present invention, the composition of the present invention may further include a PARP (Poly (ADP-ribose) polymerase) inhibitor or a pharmaceutically acceptable salt thereof as an active ingredient, but is not limited thereto.

In the present invention, when at least one miRNA gene selected from the group consisting of miR-664a-5p, miR-98-5p, and miR-95-3p is co-administered with a PARP inhibitor, a synergistic effect may be exerted, showing a significant difference compared to the control group without co-administration, but is not limited thereto. Specifically, in one embodiment of the present invention, when miR-664a-5p is co-administered with a PARP inhibitor, a synergistic effect was observed, resulting in a significant reduction in tumor growth compared to the control group without co-administration. More specifically, in one embodiment of the present invention, when miR-664a-5p is co-administered with olaparib (combination therapy), a synergistic effect was observed, resulting in a significant reduction in tumor growth compared to the control group without co-administration.

In one embodiment of the present invention, the PARP inhibitor of the present invention may be at least one selected from the group consisting of olaparib, talazoparib, niraparib, rucaparib, veliparib, and pamiparib, but is not limited thereto. Specifically, the PARP inhibitor of the present invention may be at least one selected from the group consisting of olaparib, talazoparib, niraparib, and rucaparib, but is not limited thereto.

The present invention provides a pharmaceutical composition for the prevention or treatment of cancer, including the following as active ingredients:

(i) at least one miRNA gene selected from the group consisting of miR-664a-5p, miR-98-5p, and miR-95-3p; and
(ii) (ii) a PARP (Poly (ADP-ribose) polymerase) inhibitor or a pharmaceutically acceptable salt thereof.

The present invention also provides a pharmaceutical composition for the prevention or treatment of cancer, including the following as active ingredients:
(i) miR-664a-5p; and
(ii) (ii) a PARP (Poly (ADP-ribose) polymerase) inhibitor or a pharmaceutically acceptable salt thereof.

The present invention also provides a pharmaceutical composition for the prevention or treatment of cancer, including the following as active ingredients:
(i) miR-664a-5p; and
(ii) olaparib or a pharmaceutically acceptable salt thereof.

In one embodiment of the present invention, the miR-664a-5p of the present invention may be characterized by being at least one selected from the group consisting of, but is not limited to:
(a) Directly targeting FOXM1 (forkhead box M1);
(b) Binding to the 3′UTR (3′ untranslated region) of FOXM1 mRNA;
(c) Inhibiting the expression of the FOXM1 gene or the activity of the FOXM1 protein; and
(d) Inhibiting the expression of at least one gene selected from the group consisting of BRCA2 (Breast Cancer Susceptibility Gene 2), BRIP1 (BRCA1-Interacting Protein 1), EXO1 (Exonuclease 1), and RAD51 (RAD51 Recombinase). In the present invention, FOXM1 (forkhead box M1) may be a transcription factor involved in chemotherapy resistance, but is not limited thereto.

In the present invention, FOXM1 may be a known regulator of DDR (DNA damage response) genes, but is not limited thereto. In addition, FOXM1 may be a transcription factor that regulates cell cycle progression, DDR, and chemotherapy resistance in cancer. Specifically, the cancer may be prostate cancer, and FOXM1 overexpression may be associated with poor prognosis and resistance to chemotherapy and radiotherapy in prostate cancer. In addition, FOXM1 may be involved in the regulation of DDR genes such as BRCA1/2 and RAD51, potentially contributing to PARP inhibitor resistance, however, it is not limited thereto.

In the present invention, BRCA2 (Breast Cancer Susceptibility Gene 2) may have eight BRC repeats capable of binding to RAD51 and a region that binds to DNA. The DNA-binding region of BRCA2 is structured to bind both single-stranded and double-stranded DNA, enabling BRCA2 to localize to damaged DNA. In addition, through its BRC repeats, BRCA2 facilitates the delivery of RAD51 to the damaged DNA. BRCA2 is a large protein consisting of 3,418 amino acids, and is difficult to purify. Consequently, previous studies focused on specific domains of BRCA2. However, with the recent successful purification of full-length BRCA2 protein, more detailed research has been conducted. BRCA2 may function as a catalyst that facilitates the replacement of RPA with RAD51 on single-stranded DNA bound by RPA as a result of DNA resection during the homologous recombination (HR) process, thereby enabling successful homologous recombination (HR).

In the present invention, BRIP1 (BRCA1-interacting protein 1) interacts with the BRCT domain of BRCA1, and protein truncating alterations in BRIP1 may be associated with an increased risk of ovarian cancer.

EXO1 (Exonuclease 1) is known to act in cooperation with the MRN complex, and may exhibit 5′-to-3′ exonuclease activity that initiates long resection at the ends of dsDNA to generate 3′-overhangs favorable for HDR-driven repair.

RAD51 (RAD51 Recombinase) is regulated by BRCA2 in terms of its intracellular localization and DNA-binding ability and may be associated with olaparib.

In the present invention, miR-664a-5p may regulate sensitivity to PARP inhibitors by targeting and downregulating FOXM1, a known regulator of DDR genes, but is not limited thereto.

In the present invention, “miR-664a-5p characterized by binding to the 3′UTR (3′ untranslated region) of FOXM1 mRNA” may refer to “miR-664a-5p characterized by binding to the region of FOXM1 mRNA corresponding to Sequence ID No. 13,” but is not limited thereto. Specifically, “miR-664a-5p characterized by binding to the 3′UTR (3′ untranslated region) of FOXM1 mRNA” may refer to “a region of SEQ ID NO: 14 of miR-664a-5p characterized by binding to a region of SEQ ID NO: 13 of FOXM1 mRNA” but is not limited thereto.

In the present invention, at least one gene selected from the group consisting of BRCA2, BRIP1, EXO1, and RAD51 may be a target gene of DDR (DNA damage response) genes, but is not limited thereto.

In the present invention, miR-664a-5p may directly target FOXM1, a transcription factor involved in DDR and chemotherapy resistance, to enhance the sensitivity of cancer cells to a PARP inhibitor, but is not limited thereto. Specifically, the cancer cell may be a prostate cancer cell, but is not limited thereto.

In one embodiment of the present invention, the role of urinary exosomal miR-664a-5p as a potential therapeutic target in prostate cancer (PCa) was investigated, and small RNA sequencing of urinary exosomes from prostate cancer patients with different responses to PARP inhibitors revealed that miR-664a-5p was significantly upregulated in responders. Additionally, it was confirmed that the overexpression of miR-664a-5p directly targets the transcription factor FOXM1, which is involved in DNA damage repair, thereby enhancing the sensitivity of prostate cancer cells to PARP inhibitors and inducing the downregulation of DNA damage response genes. Furthermore, the combination treatment of miR-664a-5p and olaparib effectively suppressed tumor growth in a PC-3 xenograft mouse model. Therefore, the present invention suggests that urinary exosomal miR-664a-5p serves as a potential therapeutic biomarker for predicting PARP inhibitor response in prostate cancer patients. In addition, targeting FOXM1 via miR-664a-5p represents a promising strategy to enhance the efficacy of PARP inhibitors in prostate cancer treatment.

In one embodiment of the present invention, the miRNA gene of the present invention may be provided in a form loaded onto a carrier, but is not limited thereto.

In the present invention, the carrier of the present invention may refer to a liposome or a liposome composed of a cationic lipid formulation, but is not limited thereto. In one embodiment of the present invention, the carrier may be included in reagent formulations based on Lipofectamine™ RNAiMAX, but is not limited thereto.

The pharmaceutical composition for prevention or treatment of the present invention may further include a suitable carrier, excipient, and diluent conventionally used in the manufacture of pharmaceutical compositions. The excipient may be, for example, at least one selected from the group consisting of a diluent, a binder, a disintegrant, a lubricant, an adsorbent, a humectant, a film-coating material, and a controlled-release additive.

The pharmaceutical composition of the present invention may be formulated into various dosage forms according to conventional methods, including powders, granules, sustained-release granules, enteric-coated granules, liquid preparations, eye drops, elixirs, emulsions, suspensions, tinctures, lozenges, aromatic waters, lemonades, tablets, sustained-release tablets, enteric-coated tablets, sublingual tablets, hard capsules, soft capsules, sustained-release capsules, enteric-coated capsules, pills, tinctures, soft extracts, dry extracts, fluid extracts, injections, capsules, perfusions, ointments, lotions, pastes, sprays, inhalants, patches, sterile injectable solutions, or aerosols for external use. Additionally, the external preparations may be formulated as creams, gels, patches, sprays, ointments, cataplasms, lotions, liniments, pastes, or cataplasms.

The carriers, excipients, and diluents that may be included in the pharmaceutical composition of the present invention include lactose, dextrose, sucrose, oligosaccharides, sorbitol, mannitol, xylitol, erythritol, maltitol, starch, acacia gum, alginate, gelatin, calcium phosphate, calcium silicate, cellulose, methylcellulose, microcrystalline cellulose, polyvinylpyrrolidone, water, methylparaben, propylparaben, talc, magnesium stearate, and mineral oil.

When formulating the composition, it is typically prepared using diluents or excipients such as commonly used fillers, extenders, binders, wetting agents, disintegrants, and surfactants.

In the present invention, excipients for tablets, powders, granules, capsules, pills, and troches may include fillers such as corn starch, potato starch, wheat starch, lactose, sucrose, glucose, fructose, D-mannitol, precipitated calcium carbonate, synthetic aluminum silicate, calcium phosphate monobasic, calcium sulfate, sodium chloride, sodium bicarbonate, refined lanolin, microcrystalline cellulose, dextrin, sodium alginate, methylcellulose, carboxymethylcellulose sodium, kaolin, urea, colloidal silica gel, hydroxypropyl starch, hydroxypropyl methylcellulose (HPMC), HPMC 1928, HPMC 2208, HPMC 2906, HPMC 2910, propylene glycol, casein, calcium lactate, Primojel, and the like. Binders such as gelatin, arabic gum, ethanol, agar powder, cellulose acetate phthalate, carboxymethylcellulose, carboxymethylcellulose calcium, glucose, purified water, sodium caseinate, glycerin, stearic acid, sodium carboxymethylcellulose, methylcellulose sodium, methylcellulose, microcrystalline cellulose, dextrin, hydroxycellulose, hydroxypropyl starch, hydroxymethylcellulose, purified shellac, starch paste, hydroxypropylcellulose, hydroxypropylmethylcellulose, polyvinyl alcohol, and polyvinylpyrrolidone may be used. Disintegrants may include hydroxypropyl methylcellulose, corn starch, agar powder, methylcellulose, bentonite, hydroxypropyl starch, sodium carboxymethylcellulose, sodium alginate, calcium carboxymethylcellulose, calcium citrate, sodium lauryl sulfate, anhydrous silica, 1-hydroxypropyl cellulose, dextran, ion-exchange resin, polyvinyl acetate, casein treated with formaldehyde, gelatin, alginic acid, amylose, guar gum, sodium bicarbonate, polyvinylpyrrolidone, calcium phosphate, gel-forming starch, arabic gum, amylopectin, pectin, sodium polyphosphate, ethylcellulose, sucrose, magnesium aluminum silicate, D-sorbitol solution, and hard anhydrous silica. Lubricants may include calcium stearate, magnesium stearate, stearic acid, hydrogenated vegetable oil, talc, lycopodium powder, kaolin, vaseline, sodium stearate, cacao butter, sodium salicylate, magnesium salicylate, polyethylene glycol (PEG) 4000, PEG 6000, liquid paraffin, hydrogenated soybean oil (Lubri wax), aluminum stearate, zinc stearate, sodium lauryl sulfate, magnesium oxide, macrogol, synthetic aluminum silicate, anhydrous silica, higher fatty acids, higher alcohols, silicone oil, paraffin oil, polyethylene glycol fatty acid ether, starch, sodium chloride, sodium acetate, sodium oleate, DL-leucine, and hard anhydrous silica.

In the present invention, excipients for liquid preparations may include water, diluted hydrochloric acid, diluted sulfuric acid, sodium citrate, sucrose monostearate, polyoxyethylene sorbitol fatty acid esters (Tween esters), polyoxyethylene monoalkyl ethers, lanolin ethers, lanolin esters, acetic acid, hydrochloric acid, ammonia solution, ammonium carbonate, potassium hydroxide, sodium hydroxide, prolamine, polyvinylpyrrolidone, ethylcellulose, and sodium carboxymethylcellulose.

In the present invention, syrup formulations may include sucrose solutions, other sugars, or sweeteners. Additionally, flavoring agents, coloring agents, preservatives, stabilizers, suspending agents, emulsifiers, and viscosity enhancers may be used as needed.

In the present invention, purified water may be used in emulsions, and, if necessary, emulsifiers, preservatives, stabilizers, and flavoring agents may also be included.

In the present invention, suspensions may include suspending agents such as acacia, tragacanth, methylcellulose, carboxymethylcellulose, sodium carboxymethylcellulose, microcrystalline cellulose, sodium alginate, hydroxypropyl methylcellulose (HPMC), HPMC 1828, HPMC 2906, and HPMC 2910. Additionally, if necessary, surfactants, preservatives, stabilizers, colorants, and flavoring agents may also be included.

In the present invention, injectable formulations may include solvents such as distilled water for injection, 0.9% sodium chloride injection, Ringer's injection, dextrose injection, dextrose+sodium chloride injection, polyethylene glycol (PEG), lactated Ringer's injection, ethanol, propylene glycol, non-volatile oils-sesame oil, cottonseed oil, peanut oil, soybean oil, corn oil, ethyl oleate, isopropyl myristate, and benzyl benzoate; solubilizing agents such as sodium benzoate, sodium salicylate, sodium acetate, urea, urethane, monoethylacetamide, butazolidin, propylene glycol, Tween, nicotinamide, hexamine, and dimethylacetamid; buffering agents such as weak acids and their salts (e.g., acetic acid and sodium acetate), weak bases and their salts (e.g., ammonia and ammonium acetate), organic compounds, proteins, albumin, peptones, and gums; isotonic agents such as sodium chloride; stabilizers may include sodium bisulfite (NaHSO₃), carbon dioxide gas, sodium metabisulfite (Na₂S₂O₅), sodium sulfite (Na₂SO₃), nitrogen gas (N₂), and ethylenediaminetetraacetic acid (EDTA); antioxidants such as sodium bisulfite 0.1%, sodium formaldehyde sulfoxylate, thiourea, disodium EDTA, and acetone sodium bisulfite; for pain reduction such as benzyl alcohol, chlorobutanol, procaine hydrochloride, glucose, and calcium gluconate; suspending agents such as sodium carboxymethylcellulose (CMC-Na), sodium alginate, Tween 80, and aluminum monostearate.

In the present invention, suppository bases may include cocoa butter, lanolin, Witepsol, polyethylene glycol, glycerogelatin, methylcellulose, carboxymethylcellulose, mixtures of stearic acid and oleic acid, Subanal, cottonseed oil, peanut oil, palm oil, cocoa butter+cholesterol, lecithin, Lanette wax, glyceryl monostearate, Tween or Span, Imhausen, Monolen (propylene glycol monostearate), glycerin, Adeps solidus, Butyrum Tego-G, Cebes Pharma 16, Hexaryde Base 95, Cotomar, Hydrocoat SP, S-70-XXA, S-70-XX75 (S-70-XX95), Hydrocoat 25, Hydrocoat 711, Idropostal, Massa Estrarium (A, AS, B, C, D, E, I, T), Massa-MF, Massupol, Massupol-15, Neo-Suppostal-N, Paramount-B, Supposiro (OSI, OSIX, A, B, C, D, H, L), Suppository Base IV Type (AB, B, A, BC, BBG, E, BGF, C, D, 299), Suppostal (N, Es), Weccobi (W, R, S, M, Fs), and Tigestar triglyceride base (TG-95, MA, 57).

Solid formulations for oral administration include tablets, pills, powders, granules, and capsules. These solid formulations are prepared by mixing the extract with at least one excipient, such as starch, calcium carbonate, sucrose, lactose, or gelatin. In addition to simple excipients, lubricants such as magnesium stearate talc may also be used.

Liquid formulations for oral administration include suspensions, oral solutions, emulsions, and syrups. In addition to commonly used simple diluents such as water and liquid paraffin, various excipients, such as wetting agents, sweeteners, flavoring agents, and preservatives, may be included. Formulations for parenteral administration include sterile aqueous solutions, non-aqueous solvents, suspensions, emulsions, lyophilized formulations, and suppositories. Non-aqueous solvents, suspensions may include propylene glycol, polyethylene glycol, vegetable oils such as olive oil, and injectable esters such as ethyl oleate.

The pharmaceutical composition of the present invention is administered in a pharmaceutically effective amount. In the present invention, the term “pharmaceutically effective amount” refers to an amount sufficient to treat a disease at a reasonable benefit/risk ratio applicable to medical treatment. The effective dosage level may be determined based on factors such as the type and severity of the patient's disease, the activity of the drug, the patient's sensitivity to the drug, the administration time, the route of administration, the excretion rate, the duration of treatment, concomitant medications, and other factors well known in the medical field.

The pharmaceutical composition of the present invention may be administered as a standalone therapeutic agent or in combination with other therapeutic agents. It may be administered sequentially or simultaneously with conventional therapeutic agents and may be administered as a single or multiple doses. It is important to administer an amount that achieves maximum efficacy with minimal dosage while avoiding side effects, taking all the aforementioned factors into consideration. This can be readily determined by a person skilled in the art to which the present invention pertains.

The pharmaceutical composition of the present invention can be administered to a patient through various routes. All modes of administration can be envisaged, including, for example, oral administration, subcutaneous injection, intraperitoneal injection, intravenous injection, intramuscular injection, intrathecal injection (into the perispinal space), sublingual administration, buccal administration, rectal insertion, vaginal insertion, ocular administration, otic administration, nasal administration, inhalation, spraying through the mouth or nose, topical administration to the skin, and transdermal administration.

The pharmaceutical composition of the present invention is determined based on various relevant factors, including the disease to be treated, the route of administration, the patient's age, gender, body weight, and the severity of the disease, along with the type of active ingredient used as the drug.

In the present invention, the term “patient” refers to a subject in need of disease treatment and, more specifically, includes humans or mammals such as non-human primates, mice, rats, dogs, cats, horses, and cattle.

In the present invention, the term “administration” refers to providing a predetermined composition of the present invention to a patient by any appropriate method.

In the present invention, the term “prevention” refers to any action that suppresses or delays the onset of the intended disease. The term “treatment” refers to any action in which the administration of the pharmaceutical composition according to the present invention improves or beneficially modifies the intended disease and the associated metabolic abnormalities. The term “improvement” refers to any action that reduces disease-related parameters, such as the severity of symptoms, through the administration of the composition according to the present invention.

The present invention provides a kit for the prevention or treatment of cancer, including the composition of the present invention and an instruction manual.

In one embodiment of the present invention, the kit for the prevention or treatment of cancer may be at least one selected from the group consisting of a microarray, an aptamer chip kit, an ELISA (Enzyme-Linked Immunosorbent Assay) kit, a blotting kit, an immunoprecipitation kit, an immunofluorescence assay kit, a protein chip kit, a reverse transcription polymerase chain reaction (RT-PCR) kit, and a real-time polymerase chain reaction (qRT-PCR) kit, but is not limited thereto.

In another embodiment of the present invention, the instruction manual of the present invention may provide guidance on predicting therapeutic responsiveness to a PARP (Poly (ADP-ribose) polymerase) inhibitor or prognosis based on a comparison of the expression levels of at least one miRNA gene selected from the group consisting of miR-664a-5p, miR-98-5p, and miR-95-3p. Specifically, if the expression level of the miRNA gene is higher than the expression level of the miR-664a-5p gene in a biological sample isolated from a control group, it may be predicted that the patient has high therapeutic responsiveness to a PARP inhibitor or a favorable prognosis, or if the expression level of the miRNA gene is higher than the expression level of at least one miRNA gene selected from the group consisting of miR-98-5p and miR-95-3p in a biological sample isolated from a control group, it may be predicted that the patient has low therapeutic responsiveness to a PARP inhibitor or a poor prognosis. However, the present invention is not limited thereto.

In the present invention, the kit may include a container, an instruction manual, and the like. The container may serve the function of packaging the agent and may also function to store and secure it. The material of the container may take the form of, for example, a bottle, tub, sachet, envelope, tube, or ampoule, and may be partially or entirely formed from plastic, glass, paper, foil, wax, or the like. The container may be equipped with a fully or partially removable cap, which may initially be part of the container or be attached to the container by mechanical, adhesive, or other means. Additionally, the container may be fitted with a stopper that allows access to its contents via a syringe needle. The kit may include an outer package, which may contain an instruction manual regarding the use of its components.

The present invention provides a method for treating cancer, including the following steps:
(S1) Measuring the expression level of at least one miRNA gene selected from the group consisting of miR-664a-5p, miR-98-5p, and miR-95-3p in a biological sample isolated from a cancer patient;
(S2) Comparing the expression level of the miRNA gene in step (S1) with the expression level of at least one miRNA gene selected from the group consisting of miR-664a-5p, miR-98-5p, and miR-95-3p in a biological sample isolated from a control group;
(S3) Predicting that the cancer patient has high therapeutic responsiveness to a PARP (Poly (ADP-ribose) polymerase) inhibitor or a favorable prognosis if the expression level of the miRNA gene in step (S1) is higher than the expression level of the miR-664a-5p gene in the biological sample isolated from the control group, or predicting that the cancer patient has low therapeutic responsiveness to a PARP inhibitor or a poor prognosis if the expression level of the miRNA gene in step (S1) is higher than the expression level of at least one miRNA gene selected from the group consisting of miR-98-5p and miR-95-3p in the biological sample isolated from the control group; and
(S4) Administering a PARP inhibitor for treatment if the cancer patient is predicted to have high therapeutic responsiveness to a PARP inhibitor or a favorable prognosis.

The present invention provides a method for treating cancer, including the following steps:
(S1) Measuring the expression level of at least one miRNA gene selected from the group consisting of miR-664a-5p, miR-98-5p, and miR-95-3p in a biological sample isolated from a cancer patient;
(S2) Comparing the expression level of the miRNA gene in step (S1) with the expression level of at least one miRNA gene selected from the group consisting of miR-664a-5p, miR-98-5p, and miR-95-3p in a biological sample isolated from a control group;
(S3) Predicting that the cancer patient has high therapeutic responsiveness to a PARP (Poly (ADP-ribose) polymerase) inhibitor or a favorable prognosis if the expression level of the miRNA gene in step (S1) is higher than the expression level of the miR-664a-5p gene in the biological sample isolated from the control group, or predicting that the cancer patient has low therapeutic responsiveness to a PARP inhibitor or a poor prognosis if the expression level of the miRNA gene in step (S1) is higher than the expression level of at least one miRNA gene selected from the group consisting of miR-98-5p and miR-95-3p in the biological sample isolated from the control group; and
(S4) Administering at least one miRNA gene selected from the group consisting of miR-664a-5p, miR-98-5p, and miR-95-3p, in combination with a PARP inhibitor, for treatment if the cancer patient is predicted to have high therapeutic responsiveness to a PARP inhibitor or a favorable prognosis.

Specifically, in the present invention, step (S4) may be a step of administering a PARP inhibitor and the miR-664a-5p gene for treatment if the cancer patient is predicted to have high therapeutic responsiveness to a PARP inhibitor or a favorable prognosis, but is not limited thereto.

Furthermore, the present invention provides the use of a composition including at least one miRNA gene selected from the group consisting of miR-664a-5p, miR-98-5p, and miR-95-3p as an active ingredient for predicting the prognosis of cancer.

Furthermore, the present invention provides the use of a composition including at least one miRNA gene selected from the group consisting of miR-664a-5p, miR-98-5p, and miR-95-3p as an active ingredient for manufacturing a prognostic prediction agent for cancer.

Furthermore, the present invention provides a method for predicting the prognosis of cancer, including:
(S1) Measuring the expression level of at least one miRNA gene selected from the group consisting of miR-664a-5p, miR-98-5p, and miR-95-3p in a biological sample isolated from a cancer patient;
(S2) Comparing the expression level of the miRNA gene in step (S1) with the expression level of at least one miRNA gene selected from the group consisting of miR-664a-5p, miR-98-5p, and miR-95-3p in a biological sample isolated from a control group; and
(S3) Predicting a favorable prognosis if the expression level of miR-664a-5p in step (S1) is higher than that in the biological sample isolated from the control group, or
predicting a poor prognosis if the expression level of at least one miRNA gene selected from the group consisting of miR-98-5p and miR-95-3p in step (S1) is higher than that in the biological sample isolated from the control group.

In the present invention, “when the therapeutic responsiveness to the PARP inhibitor is high or the prognosis is predicted to be favorable” may be used interchangeably with “for a cancer patient whose therapeutic responsiveness to the PARP inhibitor is high or whose prognosis is predicted to be favorable,” but is not limited thereto.

In the present invention, the “method for treating cancer” may be applied concurrently or sequentially with conventional cancer treatment methods, but is not limited thereto.

In the present invention, the “method for treating cancer” may include co-administration of a prophylactic or therapeutic pharmaceutical composition for the prevention or treatment of cancer.

In the present invention, the method for providing information for predicting therapeutic responsiveness or prognosis to a PARP inhibitor, the composition for predicting therapeutic responsiveness or prognosis to a PARP inhibitor, the kit for predicting therapeutic responsiveness or prognosis to a PARP inhibitor, the pharmaceutical composition for the prevention or treatment of cancer, the kit for the prevention or treatment of cancer, the method for screening anticancer agents, the composition for screening anticancer agents, and the kit for screening anticancer agents may be applied without limitation in cases where the aforementioned content is applicable.

In the present invention, the terms used have been selected as widely used general terms while considering their functions in the invention. However, these terms may vary depending on the intent of a person skilled in the art, legal precedents, or the emergence of new technologies. Additionally, in certain cases, terms may have been arbitrarily selected by the applicant. In such instances, their meanings will be described in detail in the relevant sections of the specification. Therefore, the terms used in the present invention should be defined based on their meanings and the overall content of the invention, rather than merely their nominal designations.

Throughout the entire specification of the present invention, when a certain part is described as “comprising” a certain component, it means that, unless otherwise explicitly stated, other components may be further included rather than being excluded. Throughout the entire specification of the present invention, terms such as “approximately” or “substantially” are used to indicate values close to or within the inherent manufacturing and material tolerances specified for the stated meaning. These terms are employed to aid in the understanding of the present invention and to prevent unscrupulous infringers from unfairly exploiting the disclosed content, which may include precise or absolute numerical values.

Throughout the entire specification of the present invention, the term “a combination thereof” included in Markush-type expressions refers to a mixture or combination of one or more selected from the group consisting of the components described in the Markush-type expression. It is intended to mean that one or more selected from the group consisting of the stated components are included.

Hereinafter, preferred embodiments are presented to aid in the understanding of the present invention. However, the following embodiments are provided merely to facilitate a better understanding of the present invention and are not intended to limit the scope of the present invention.

### EXAMPLES

### Patients

The study protocol was approved and carried out in accordance with the approved guidelines by the Institutional Review Board at the Catholic University of Korea, Seoul St. Mary's Hospital (IRB approval No. MC20SNSI0161). Urine specimens of PCa patients who different responses to PARP inhibitors were obtained from the Korea Prostate Bank (Seoul, Republic of Korea) with informed consent. Mid-stream urine specimens were collected before drug treatment, and were centrifuged at 2,500 rpm for 20 min at 4° C. The supernatant was transferred to new tubes and stored at −80° C.

### Small RNA Sequencing

Urinary exosome isolation, exosomal RNA extraction, library preparation, sequencing, and data analysis were performed as described in our previous study (Sci Rep. 2021 Apr. 1;11 (1):7355. doi: 10.1038/s41598-021-86785-z. PMID: 33795765; PMCID: PMC8016942). Urinary exosomes were isolated using ATPS (Ex02D™, ExosomePlus, Gyeonggi, Republic of Korea) and exosomal total RNA was extracted using a miRNeasy Serum/Plasma Kit (Qiagen, Hilden, Germany). NEBNext® Multiplex Small RNA Library Prep kit (New England BioLabs, Ipswich, MA, USA) was used for library construction, and high-throughput sequencing was performed on a NextSeq550 system (Illumina, San Diego, CA, USA). Sequence reads were mapped using Bowtie 2, and read counts were normalized using CPM+TMM method for comparison between samples.

### Drugs and Reagent

Olaparib (AZD2281, S1060), talazoparib (BMN 673, S7048), niraparib (MK-4827, S2741), and rucaparib (AG-014669, S1098) were purchased from Selleck Chemicals LLC (Houston, TX, USA). Dimethyl sulfoxide (DMSO) and (2-hydroxypropyl)-β were purchased from Sigma-Aldrich (St. Louis, MO, USA). For in vitro experiments, all drugs were dissolved in DMSO and stored at −20° C. The final DMSO concentration did not exceed 0.1%.

miR-664a-5p mimic (Assay ID: MC13213), miR-98-5p mimic (Assay ID: MC10426), miR-95-3p mimic (Assay ID: MC10147), and miRNA negative control (NC) mimic were purchased from Thermo Fisher Scientific Inc. (Waltham, MA, USA).

### Cell Lines and Cell Culture

C4-2B and 22Rv1 cells were obtained from the American Type Culture Collection® (Manassas, VA, USA), and PC-3 cells were obtained from Korean Cell Line Bank (Seoul, Republic of Korea). All of three cell lines were cultured in RPMI 1640 medium (Gibco; Thermo Fisher Scientific, Inc.) supplemented with 10% fetal bovine serum (FBS; Gibco), 100 units/mL penicillin, and 0.1 mg/mL streptomycin (Gibco). Cultures were incubated at 37° C. in a humidified atmosphere containing 5% CO₂. All cells are routinely tested and checked for the absence of mycoplasma.

### miRNA Transfection and Treatment

The cells were transfected with miR-664a-5p, miR-98-5p, miR-95-3p, and miR-NC using Lipofectamine™ RNAiMAX (Invitrogen; Thermo Fisher Scientific, Inc) according to manufacturer's protocol. After 5 hours of incubation, the medium was replaced, and the cells were treated with PARP inhibitors or DMSO. The concentration of PAPR inhibitor applied were as follows: for C4-2B cells, 2 μM olaparib, 20 nM talazoparib, 2 μM niraparib, and 10 μM rucaparib; for PC-3 cells, 5 μM olaparib and 0.2 μM talazoparib; and for 22Rv1 cells, 20 μM olaparib and 0.5 μM talazoparib.

After the indicated time period, the cells were harvested and used for cellular and molecular analysis.

### Cell Viability Assay

Cell viability was assessed using cell counting kit-8 (CCK-8, Dojindo Laboratories, Rockville, MD, USA) according to the manufacturer's instructions. After 5 days of transfection and treatment, supernatant was replaced by culture medium containing 10% CCK-8 reagent and cultured for additional 1 hour. Cell viability was indicated by the absorbance at 450 nm using a microplate reader (Molecular Devices, San Jose, CA, USA). All experiments were performed in triplicate.

### Reverse Transcription-Quantitative Polymerase Chain Reaction (qRT-PCR)

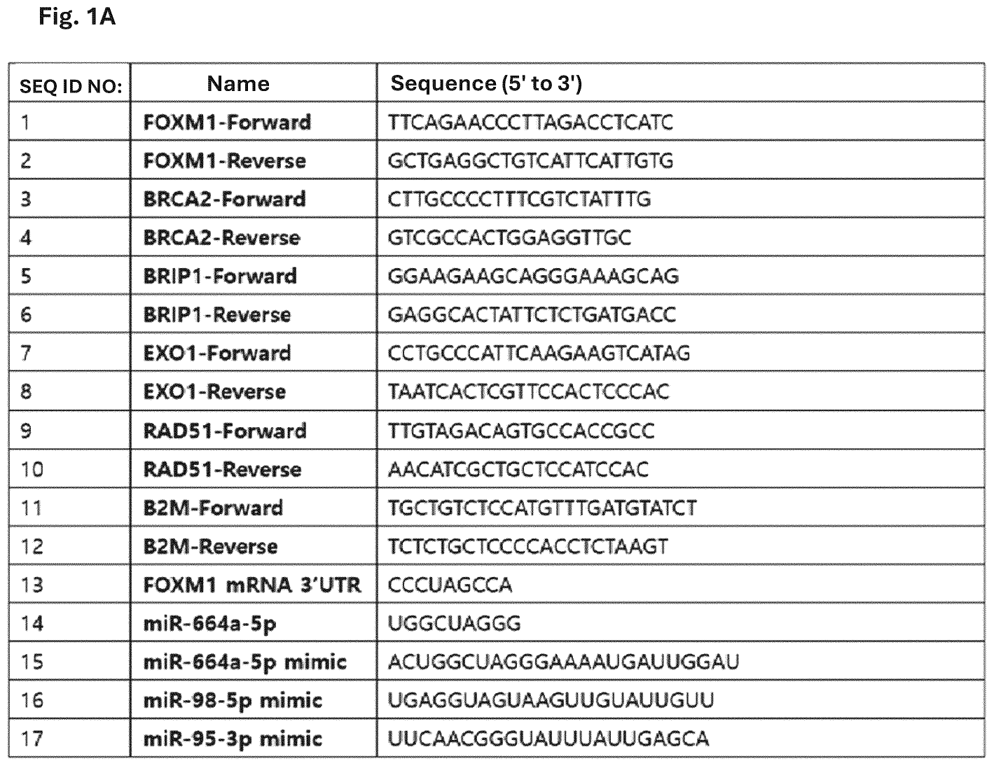

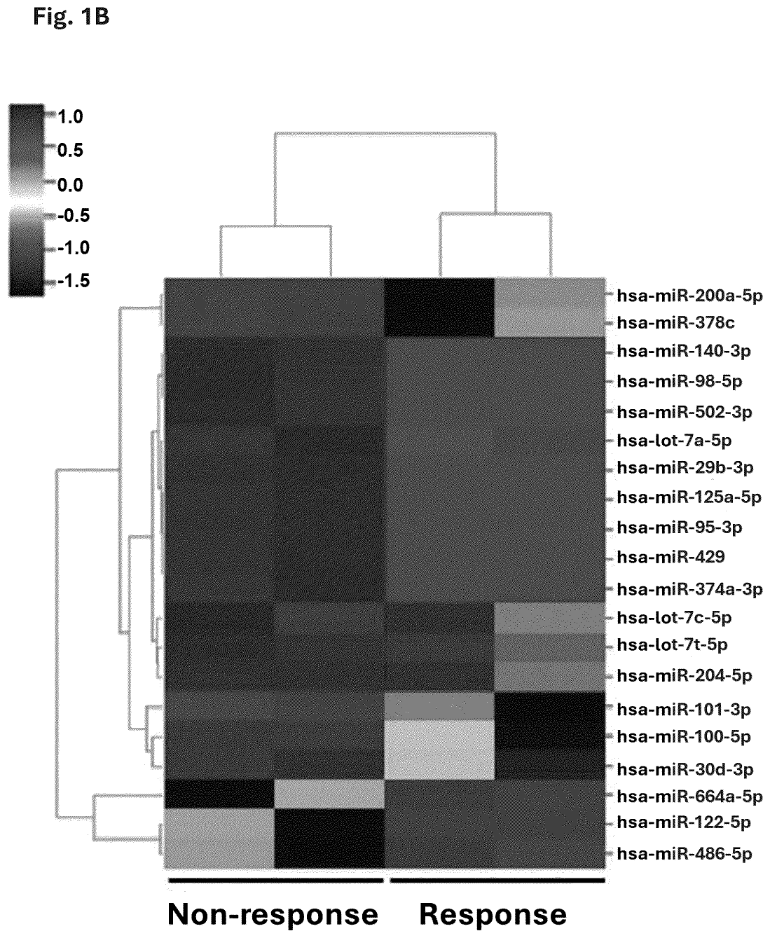

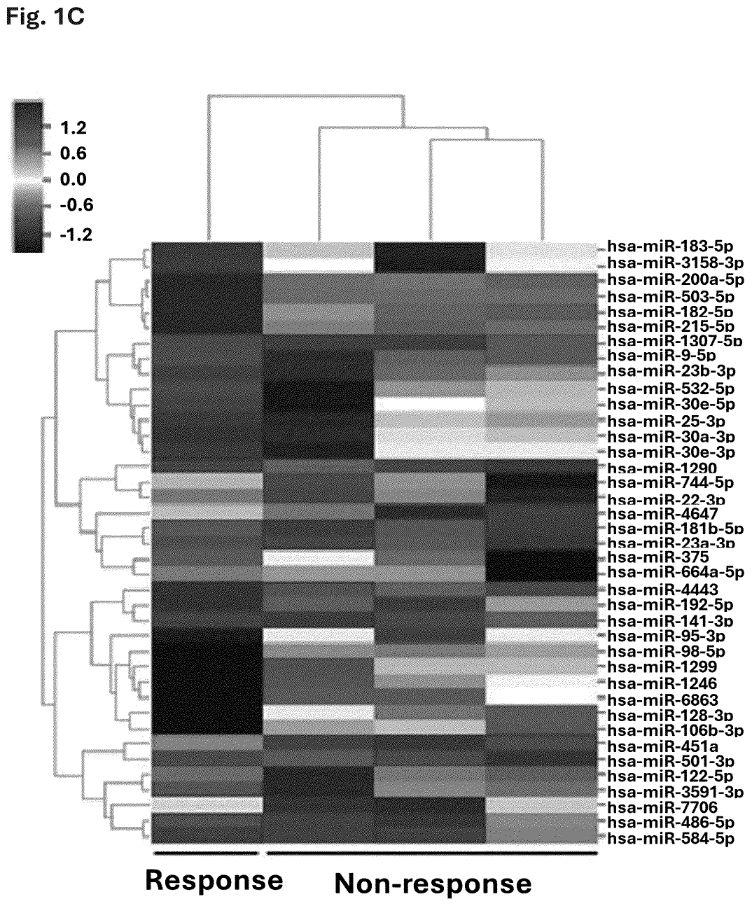

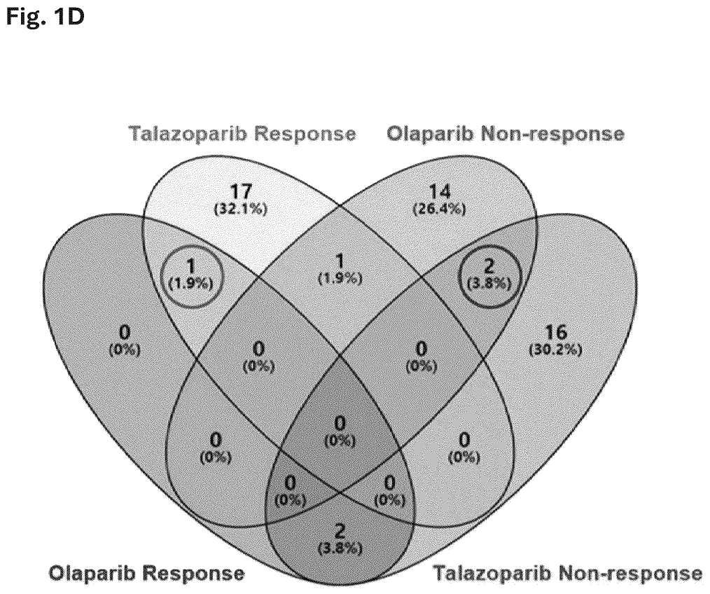

After 3 days of transfection and treatment, RNA extraction, cDNA synthesis and qPCR were carried out as previously described (2021 Jun. 15;11(6):2944-2959. PMID: 34249437; PMCID: PMC8263667). Relative gene expression was determined by normalizing to B2M using the 2^{-ΔΔCT} method. Primer pairs used are listed in FIG. 1A.

### Western Blot Analyses

Cell lysates were prepared with PRO-PREP™ protein extraction solution (iNtRON Biotechnology Inc., Gyeonggi, Republic of Korea). Proteins were separated by SDS-PAGE and transferred onto nitrocellulose membranes. The membranes were blocked and subsequently incubated overnight at 4° C. with specific primary antibodies against FOXM1 (1:1000, Santa Cruz Biotechnology, Inc., Dallas, TX, USA) and β-actin (1:5000, Abcam Limitied, Cambridge, UK). The membranes were washed and then incubated with a horseradish peroxidase-conjugated horse anti-mouse or anti-rabbit IgG (GenDEPOT, Barker, TX, USA). The protein bands were visualized using an enhanced chemiluminescence detection kit (Thermo Fisher Scientific, Inc.), and detected using X-ray film (2021 Jun. 15;11 (6): 2944-2959. PMID: 34249437; PMCID: PMC8263667).

### Dual-Luciferase Reporter Assays

The 3′UTR sequence of FOXM1 mRNA including predicted target site of miR-664a-5p were chemically synthesized from Bioneer Corporation (Daejeon, Republic of Korea). Synthesized DNA fragments were inserted into the NheI and XbaI sites of pmirGLO (Promega Corporation, Madison, WI, USA), and identified by DNA sequencing assay. PC-3 cells were co-transfected with reporter plasmid and miRNA in 24-well plates using Lipofectamine® 2000 transfection reagent (Invitrogen). Luciferase activity was measured after incubation for 48 hours using the Dual-Luciferase® Reporter Assay System (Promega Corp.) according to the manufacturer's instructions. Firefly luciferase activity was normalized to renilla luciferase activity.

### In Vivo Study

All experimental procedures were approved by the Institutional Animal Care and Use Committee of the Catholic University of Korea (CUMC-2022-0109-01). Five-week-old male BALB/c nude mice (body weight 20-25 g) were obtained from Orient Bio Inc. (Gyeonggi, Republic of Korea). 1.5×10⁶ PC-3 cells were subcutaneously injected into the flank of the mouse in 100 μL of PBS mixed with 100 μL of Matrigel® (Corning®, NY, USA). When tumor volumes reached approximately 40 mm³, the mice were randomly divided into four groups (n=6): (1) control group (vehicle+miR-NC); (2) olaparib group (olaparib+miR-NC); (3) miR-664a-5p group (vehicle+miR-664a-5p); (4) combination group (olaparib+miR-664a-5p).

For the in vivo experiments, olaparib was dissolved in 10% (2-hydroxypropyl)-β-cyclodextrin at a concentration of 50 mg/kg and administered via intraperitoneal injections 5 days a week. In vivo jetPEI® (Polyplus Transfection®, Illkirch, France) was used as an in vivo delivery agent for miRNAs. The miRNA/in vivo-jetPEI® complexes (miRNA 10 μg and in vivo-jetPEI® reagent 1.2 μl for each mouse) was delivered intratumorally three times at 2-day intervals. Tumor size was measured through the experimental period at 3-day intervals, and tumor volume was calculated as width²×length×0.5. The mouse was sacrificed after 10 days of treatment. Mouse xenografts tissue was embedded in paraffin wax for immunohistochemical (IHC) assay using antibodies against Ki-67 (1:300, Abcam Limited). Counter staining with hematoxylin was performed. Tissue sections were examined under a light microscope (Carl Zeiss Inc., Jena, Germany).

### Statistical Analyses

The results of in vitro experiments were expressed as mean±SD (standard deviation), and the results of in vivo experiments were expressed as mean±SE (standard error). Differences between values were analyzed using Student's t test or one-way analysis of variance (ANOVA) test in GraphPad Prism 5 (GraphPad Software, San Diego, CA, USA). P-values<0.05 were considered statistically significant.

### Example 1. Identification of Urinary Exosomal miRNAs Associated With PARP Inhibitor Response in PCa Patients

We performed small RNA sequencing on urine samples from PCa patients (n=8) who exhibited different responses to olaparib and talazoparib. Hierarchical clustering analysis revealed distinct miRNA expression profiles between the response and non-response groups for both olaparib (FIG. 1B) and talazoparib (FIG. 1C). MiR-664a-5p was significantly upregulated in the response group for both PARP inhibitors, while miR-98-5p and miR-95-3p were upregulated in the non-response group (FIG. 1D).

This suggests that the differential expression of miR-664a-5p, miR-98-5p, and miR-95-3p between the response group and non-response group to a PARP inhibitor plays a crucial role in regulating the efficacy of the PARP inhibitor.

### Example 2. MiR-664a-5p Enhances PARP Inhibitor Sensitivity in Prostate Cancer Cells

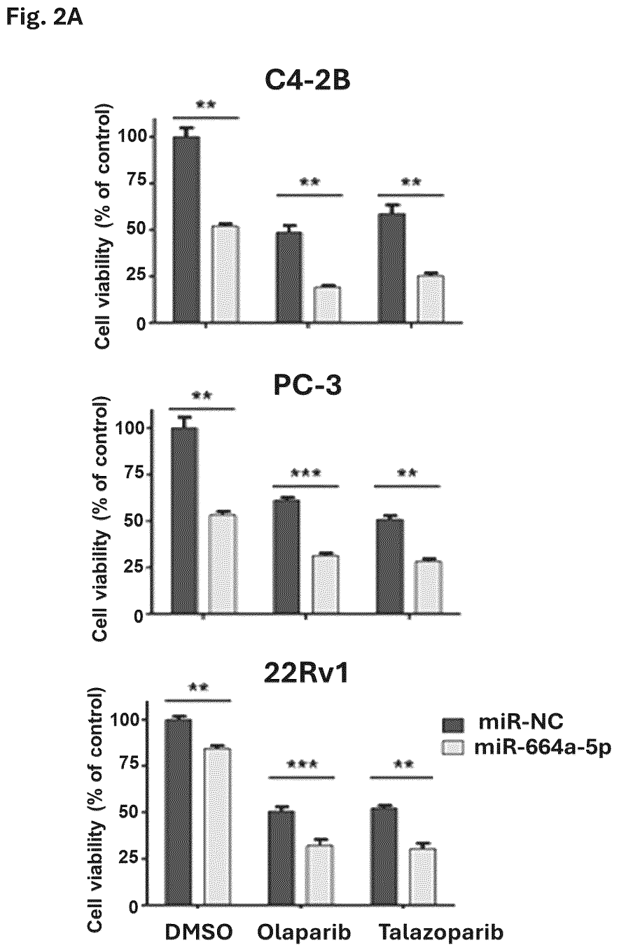

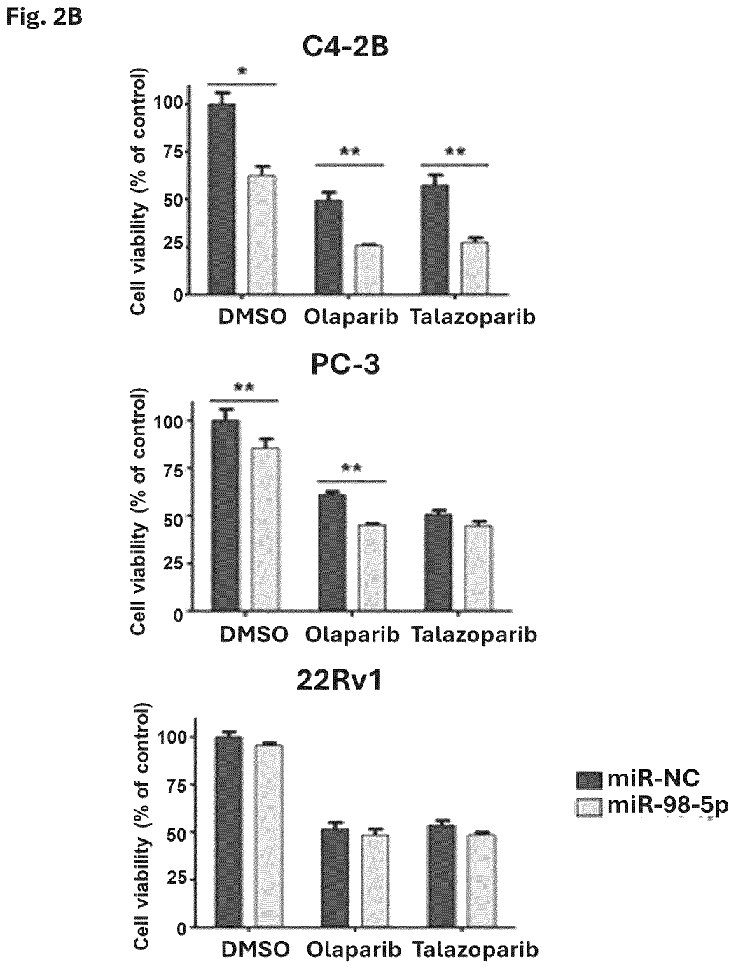

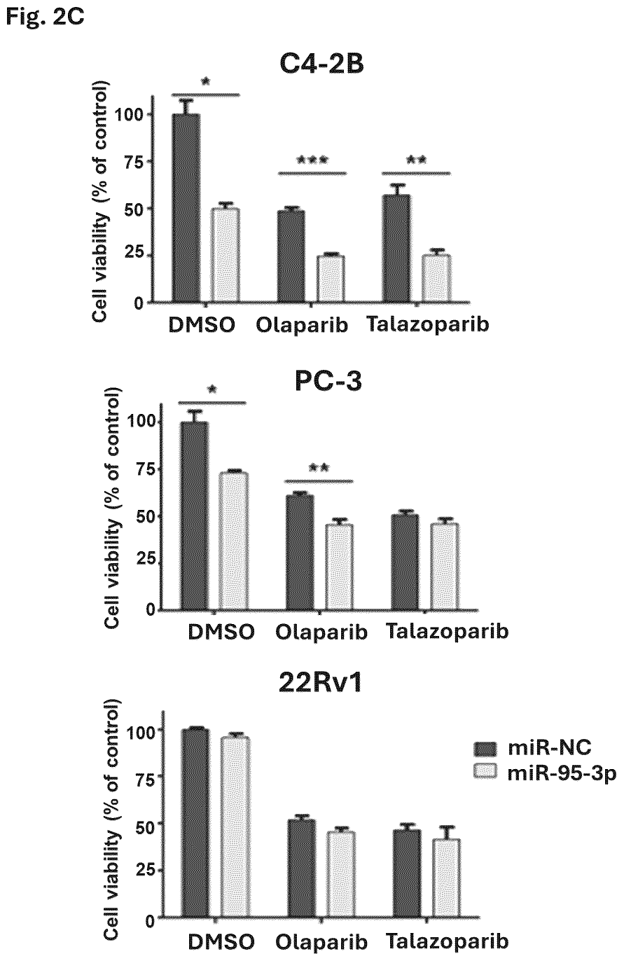

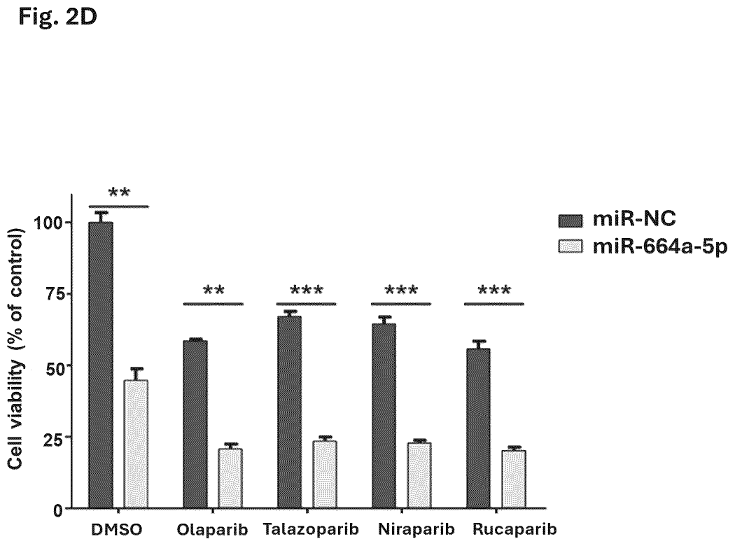

To validate the functional role of the identified miRNAs in PARP inhibitor response, we transfected prostate cancer cell lines (C4-2B, PC-3, and 22Rv1) with miRNA mimics and assessed their sensitivity to olaparib and talazoparib using cell viability assays. Overexpression of miR-664a-5p significantly increased the sensitivity of all three cell lines to PARP inhibitors (FIG. 2A). Overexpression of miR-98-5p and miR-95-3p significantly increased the sensitivity of C4-2B cells to the PARP inhibitor. In contrast, overexpression of miR-98-5p and miR-95-3p in PC-3 cells increased sensitivity to olaparib but had no effect on talazoparib. In 22Rv1 cells, the overexpression of miR-98-5p and miR-95-3p did not affect sensitivity to either PARP inhibitor (FIG. 2B and FIG. 2C).

Overexpression of miR-664a-5p significantly enhanced the sensitivity of C4-2B cells to all four PARP inhibitors (FIG. 2D). These suggest that miR-664a-5p plays an important role in modulating the response to various PARP inhibitors in prostate cancer cells.

### Example 3. miR-664a-5p Downregulates FOXM1 Expression by Directly Targeting its 3′UTR

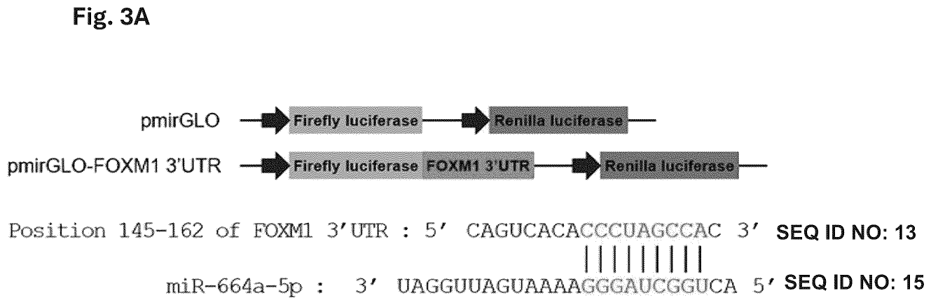

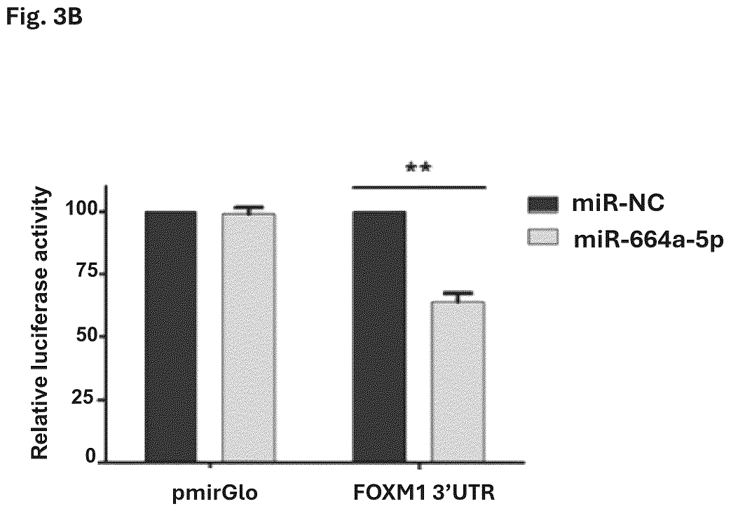

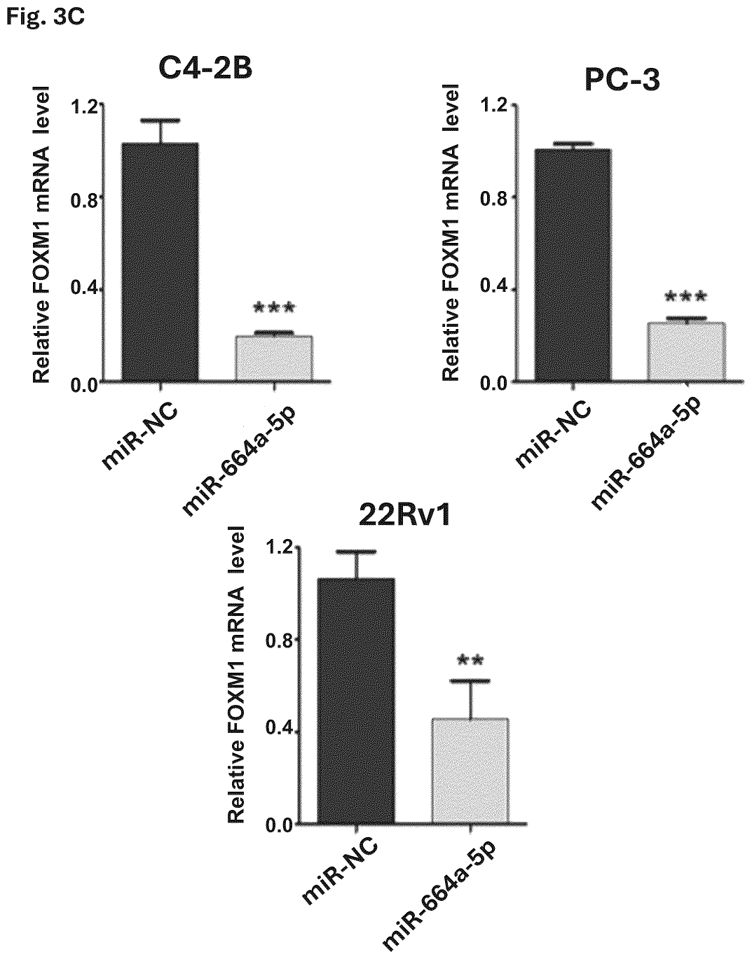

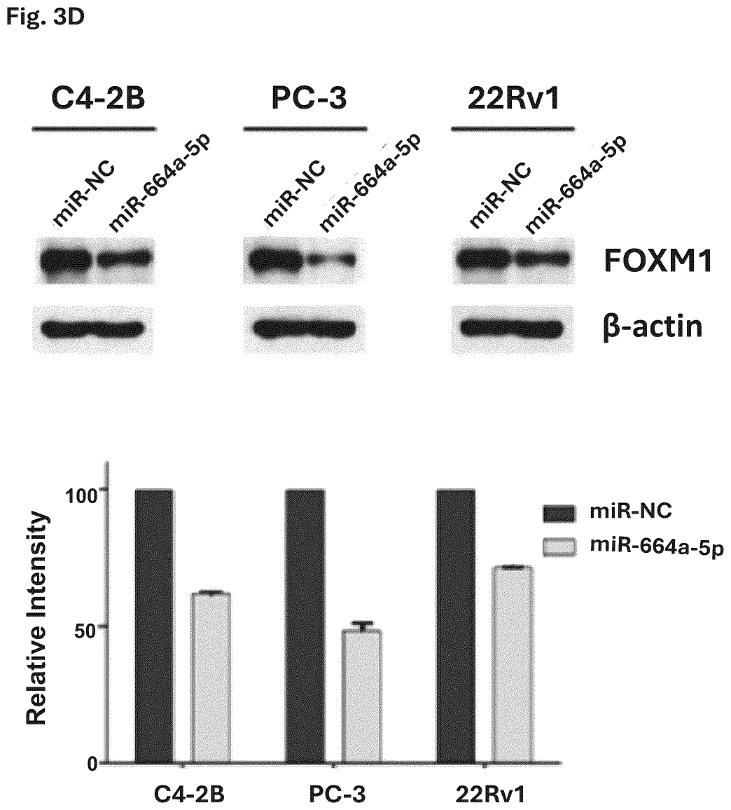

To identify the target genes of miR-664a-5p, we explored the miRWalk database and found a putative binding site for miR-664a-5p in the 3′UTR of FOXM1 mRNA. We investigated whether miR-664a-5p regulates FOXM1 expression in prostate cancer cells. The predicted binding site between miR-664a-5p and FOXM1 3′UTR is shown in FIG. 3A. Dual-luciferase® reporter assays using a vector containing the 3′UTR of FOXM1 showed that miR-664a-5p significantly suppressed the luciferase activity of the FOXM1 3′UTR construct (FIG. 3B). This indicates that miR-664a-5p directly binds to the 3′UTR of FOXM1 to downregulate its expression. Overexpression of miR-664a-5p significantly reduced FOXM1 mRNA (FIG. 3C) and protein levels (FIG. 3D) in C4-2B, PC-3, and 22Rv1 cells.

### Example 4. miR-664a-5p and Olaparib Synergistically Suppress DDR Gene Expression

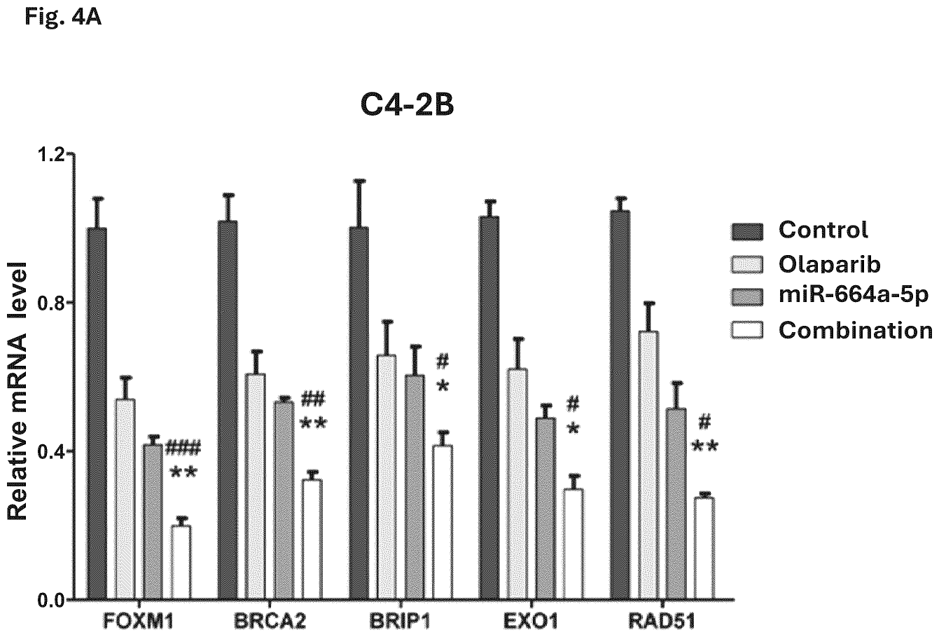

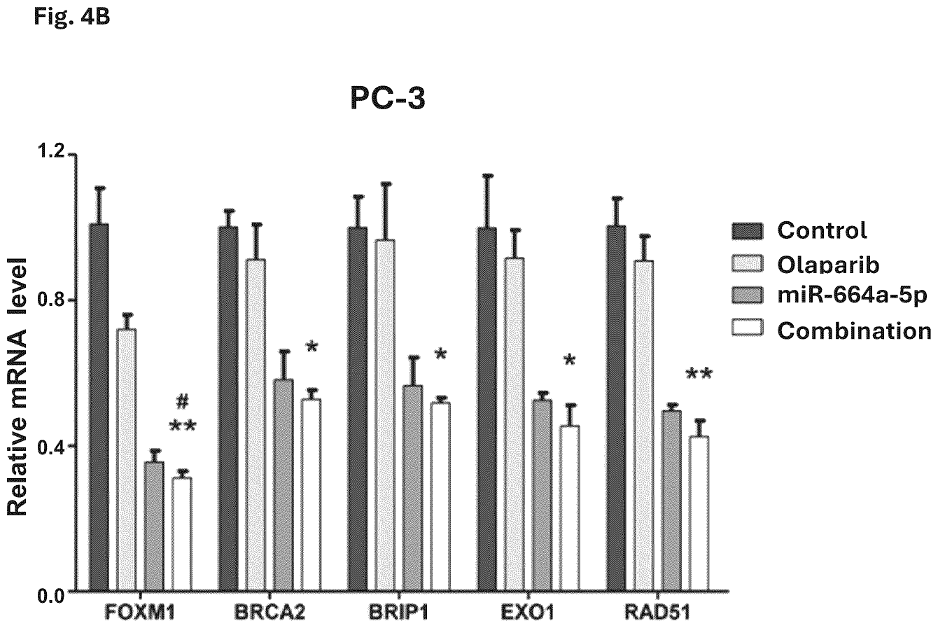

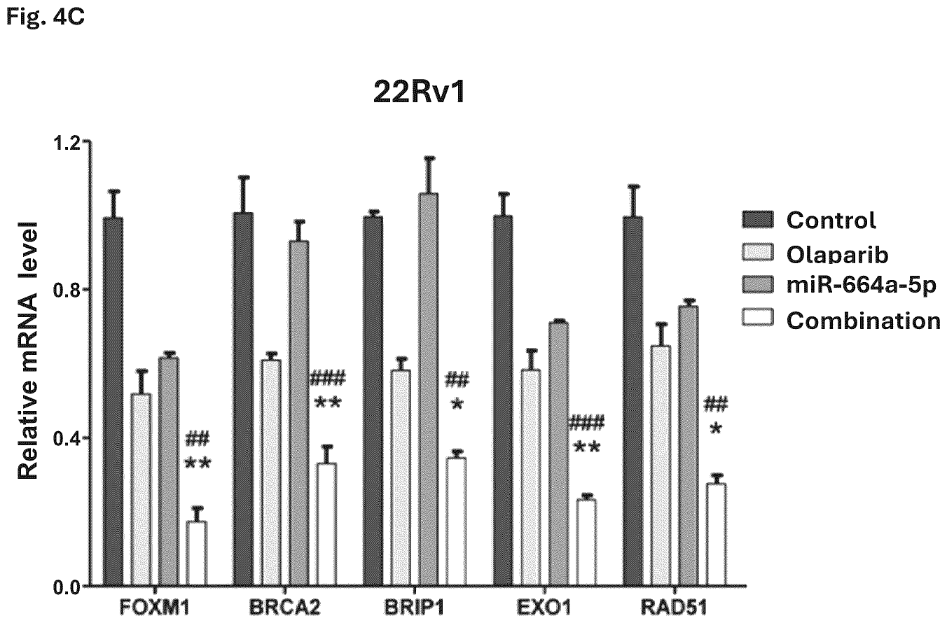

The expression of four genes (BRCA2, BRIP1, EXO1, and RAD51), which are major DDR genes and targets of FOXM1, was examined in prostate cancer cells treated with miR-664a-5p, olaparib, or a combination thereof. When miR-664a-5p and olaparib were administered in combination, the expression levels of the four DDR genes were significantly reduced compared to treatment with either agent alone (FIGS. 4A to 4C).

Through Examples 3 and 4 of the present invention, it was confirmed that miR-664a-5p of the present invention directly targets FOXM1, a transcription factor involved in DDR and chemoresistance, and enhances the sensitivity of prostate cancer cells to PARP inhibitors by downregulating FOXM1 and its downstream genes.

### Example 5. Combination Treatment of miR-664a-5p and Olaparib Effectively Suppresses Tumor Growth of Prostate Cancer in Vivo

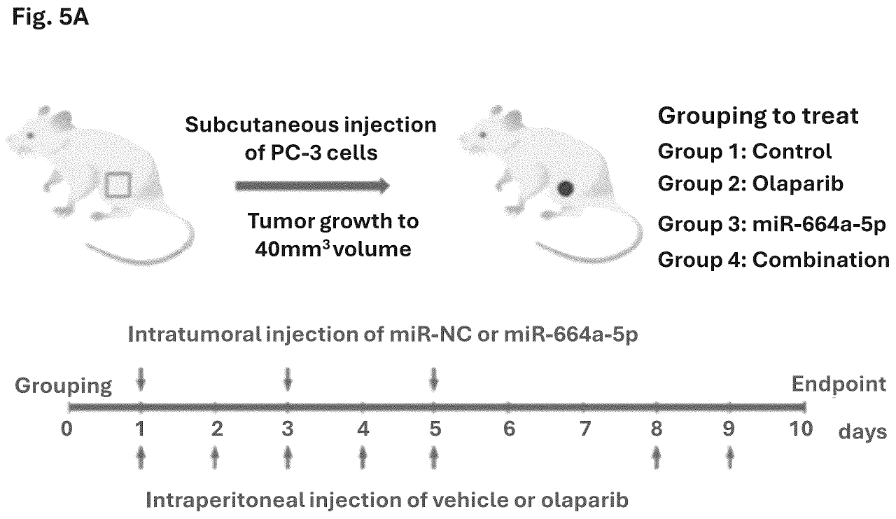

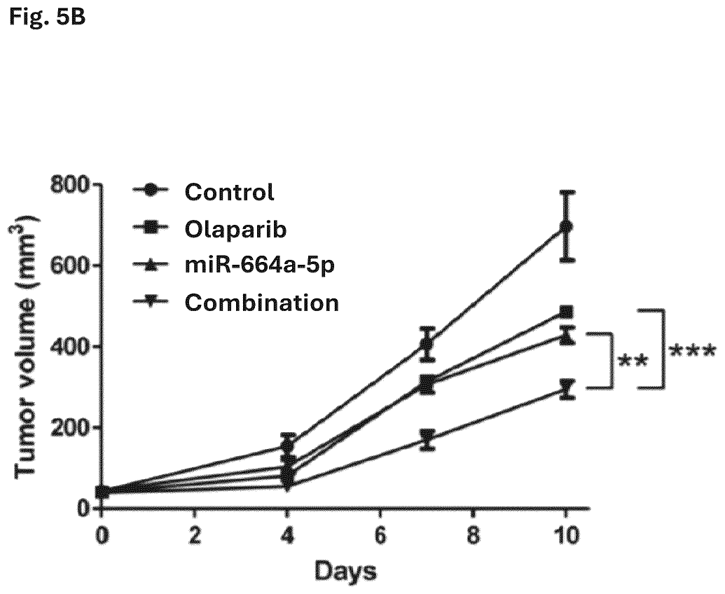

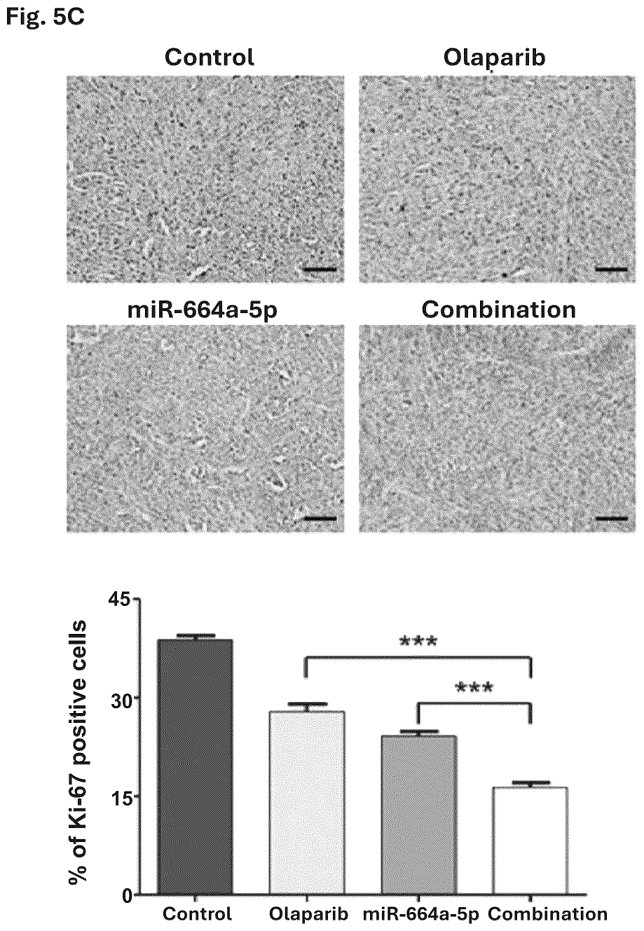

We evaluated the efficacy of combination treatment of miR-664a-5p and olaparib in a PC-3 xenograft mouse model (FIG. 5A). Mouse treated with the combination therapy exhibited significantly reduced tumor growth compared to those treated with either miR-664a-5p or olaparib alone (FIG. 5B). IHC analysis of the xenograft tumors revealed a significant decrease in Ki-67 positive cells in the combination treatment group (FIG. 5C).

The combined treatment of miR-664a-5p and olaparib effectively inhibited tumor growth of prostate cancer in vivo, suggesting that miR-664a-5p can be used as a therapeutic agent in combination with a PARP inhibitor. Furthermore, it provides a basis for developing miRNA-based therapies in combination with PARP inhibitors for cancer treatment.

The foregoing description of the present invention is provided by way of example, and it will be understood by those skilled in the art that various modifications can be made in other specific forms without altering the technical spirit or essential characteristics of the present invention. Therefore, the embodiments described above should be understood as illustrative in all respects and not as limiting.

1-24. (canceled)

25. A method for providing information for predicting therapeutic responsiveness or prognosis for a PARP (Poly (ADP-ribose) polymerase) inhibitor, comprising:
(S1) Measuring the expression level of at least one miRNA gene selected from the group consisting of miR-664a-5p, miR-98-5p, and miR-95-3p from a biological sample isolated from a cancer patient;
(S2) Comparing the expression level of the miRNA gene measured in step (S1) with the expression level of at least one miRNA gene selected from the group consisting of miR-664a-5p, miR-98-5p, and miR-95-3p in a biological sample isolated from a control group; and
(S3) Predicting that the therapeutic responsiveness to a PARP inhibitor is high, or the prognosis is favorable, when the expression level of the miRNA gene measured in step (S1) is higher than the expression level of miR-664a-5p in the biological sample isolated from the control group, or predicting that the therapeutic responsiveness to a PARP inhibitor is low, or the prognosis is poor, when the expression level of the miRNA gene measured in step (S1) is higher than the expression level of at least one miRNA gene selected from the group consisting of miR-98-5p and miR-95-3p in the biological sample isolated from the control group.

26. The method of claim 25, wherein the method further comprises, prior to step (S1), the steps of:
(a) administering the PARP inhibitor to the cancer patient; and
(b) extracting the biological sample from the cancer patient.

27. The method of claim 25, wherein the method further comprises, after step (S3), a step of determining to treat a patient predicted to have high therapeutic responsiveness or a favorable prognosis with the PARP inhibitor.

28. The method of claim 25, wherein the cancer in step (S1) is prostate cancer.

29. The method of claim 25, wherein the biological sample in step (S1) is at least one selected from the group consisting of urine, urine-derived exosomes, feces, feces-derived exosomes, saliva, and saliva-derived exosomes.

30. The method of claim 25, wherein the PARP inhibitor is at least one selected from the group consisting of olaparib, talazoparib, niraparib, and rucaparib.

31. A kit for predicting therapeutic responsiveness or prognosis for a PARP inhibitor, comprising an instruction manual, and a composition comprising an agent for measuring the expression level of at least one miRNA gene selected from the group consisting of miR-664a-5p, miR-98-5p, and miR-95-3p as an active ingredient.

32. The kit of claim 31, wherein the agent for measuring the expression level of the miRNA gene is at least one agent selected from the group consisting of primer and probe that specifically bind to the gene.

33. The kit of claim 31, wherein the PARP inhibitor is at least one selected from the group consisting of olaparib, talazoparib, niraparib, and rucaparib.

34. The kit of claim 31, wherein the instruction manual states:
that a cancer patient whose expression level of the miR-664a-5p gene is higher than the expression level of the miR-664a-5p gene in a biological sample isolated from a control group is predicted to have high therapeutic responsiveness to a PARP (Poly (ADP-ribose) polymerase) inhibitor or a favorable prognosis; or
that a cancer patient whose expression level of at least one miRNA gene selected from the group consisting of miR-98-5p and miR-95-3p is higher than the expression level of at least one miRNA gene selected from the group consisting of miR-98-5p and miR-95-3p in a biological sample isolated from a control group is predicted to have low therapeutic responsiveness to a PARP inhibitor or a poor prognosis.

35. The kit of claim 34, wherein the cancer is prostate cancer.

36. The kit of claim 31, wherein the kit is at least one selected from the group consisting of a microarray, an aptamer chip kit, an ELISA (Enzyme-Linked Immunosorbent Assay) kit, a blotting kit, an immunoprecipitation kit, an immunofluorescence assay kit, a protein chip kit, a reverse transcription polymerase chain reaction (RT-PCR) kit, and a real-time polymerase chain reaction (qRT-PCR) kit.

37. A method for preventing or treating cancer, comprising administering a pharmaceutical composition comprising at least one miRNA gene selected from the group consisting of miR-664a-5p, miR-98-5p, and miR-95-3p, or an overexpression agent thereof as an active ingredient to a subject in need thereof.

38. The method of claim 37, wherein the cancer is prostate cancer.

39. The method of claim 37, wherein the composition further comprises a PARP (Poly (ADP-ribose) polymerase) inhibitor, or a pharmaceutically acceptable salt thereof as an active ingredient, and wherein the PARP inhibitor is at least one selected from the group consisting of olaparib, talazoparib, niraparib, and rucaparib.

40. The method of claim 37, wherein the miR-664a-5p is characterized by at least one selected from the group consisting of:
(a) directly targeting FOXM1 (forkhead box M1);
(b) binding to the 3′UTR (3′ untranslated region) of FOXM1 mRNA;
(c) inhibiting the expression of the FOXM1 gene or the activity of the FOXM1 protein; and
(d) inhibiting the expression of at least one gene selected from the group consisting of BRCA2 (Breast Cancer Susceptibility Gene 2), BRIP1 (BRCA1-Interacting Protein 1), EXO1 (Exonuclease 1), and RAD51 (RAD51 Recombinase).

41. The method of claim 37, wherein the miRNA gene is provided in a form loaded onto a carrier.

42. A method for screening an anticancer agent, comprising:
(S1) Treating a biological sample isolated from a cancer patient with a test substance;
(S2) Measuring the expression level of at least one miRNA gene selected from the group consisting of miR-664a-5p, miR-98-5p, and miR-95-3p in the biological sample treated with the test substance; and
(S3) Screening a test substance that increases the expression level of miR-664a-5p compared to a control sample or screening a test substance that decreases the expression level of at least one miRNA gene selected from the group consisting of miR-98-5p and miR-95-3p compared to a control sample.

43. The method for screening an anticancer agent of claim 42, wherein the cancer in step (S1) is prostate cancer.

44. The method for screening an anticancer agent of claim 42, wherein the biological sample in step (S1) is at least one selected from the group consisting of urine, urine-derived exosomes, feces, feces-derived exosomes, saliva, and saliva-derived exosomes.


SEQUENCE LISTING (17 sequences)

SEQ ID NO: 1 - DNA, 22 bp, synthetic construct
  source 1..22: mol_type = other DNA
ttcagaacccttagacctcatc

SEQ ID NO: 2 - DNA, 22 bp, synthetic construct
  source 1..22: mol_type = other DNA
gctgaggctgtcattcattgtg

SEQ ID NO: 3 - DNA, 21 bp, synthetic construct
  source 1..21: mol_type = other DNA
cttgcccctttcgtctatttg

SEQ ID NO: 4 - DNA, 18 bp, synthetic construct
  source 1..18: mol_type = other DNA
gtcgccactggaggttgc

SEQ ID NO: 5 - DNA, 20 bp, synthetic construct
  source 1..20: mol_type = other DNA
ggaagaagcagggaaagcag

SEQ ID NO: 6 - DNA, 22 bp, synthetic construct
  source 1..22: mol_type = other DNA
gaggcactattctctgatgacc

SEQ ID NO: 7 - DNA, 23 bp, synthetic construct
  source 1..23: mol_type = other DNA
cctgcccattcaagaagtcatag

SEQ ID NO: 8 - DNA, 22 bp, synthetic construct
  source 1..22: mol_type = other DNA
taatcactcgttccactcccac

SEQ ID NO: 9 - DNA, 20 bp, synthetic construct
  source 1..20: mol_type = other DNA
ttgtagacagtgccaccgcc

SEQ ID NO: 10 - DNA, 20 bp, synthetic construct
  source 1..20: mol_type = other DNA
aacatcgctgctccatccac

SEQ ID NO: 11 - DNA, 25 bp, synthetic construct
  source 1..25: mol_type = other DNA
tgctgtctccatgtttgatgtatct

SEQ ID NO: 12 - DNA, 22 bp, synthetic construct
  source 1..22: mol_type = other DNA
tctctgctccccacctctaagt

SEQ ID NO: 13-14 - skipped (2 entries)

SEQ ID NO: 15 - RNA, 24 nt, synthetic construct
  source 1..24: mol_type = other RNA
actggctagggaaaatgattggat

SEQ ID NO: 16 - RNA, 22 nt, synthetic construct
  source 1..22: mol_type = other RNA
tgaggtagtaagttgtattgtt

SEQ ID NO: 17 - RNA, 22 nt, synthetic construct
  source 1..22: mol_type = other RNA
ttcaacgggtatttattgagca